(19) United States
(12) Patent Application Publication
(10) Pub. No.: US 2024/0408004 A1
(43) Pub. Date: Dec. 12, 2024

(54) COMPOSITIONS, DEVICES, AND METHODS FOR THE TREATMENT OF OVERDOSE AND REWARD-BASED DISORDERS

(71) Applicants: Aegis Therapeutics, LLC, San Diego, CA (US); Indivior UK Limited, Hull (GB)

(72) Inventors: Edward T. MAGGIO, San Diego, CA (US); Roger CRYSTAL, Santa Monica, CA (US); Mark ELLISON, San Diego, CA (US); Phil SKOLNICK, Santa Monica, CA (US); Arvind AGRAWAL, Santa Monica, CA (US)

(73) Assignees: Aegis Therapeutics, LLC, San Diego, CA (US); Indivior UK Limited, Hull (GB)

(21) Appl. No.: 18/392,013

(22) Filed: Dec. 21, 2023

Related U.S. Application Data
(63) Continuation of application No. 16/721,872, filed on Dec. 19, 2019, now abandoned.
Application No. 16/721,872 is a continuation-in-part of application No. 16/311,944, filed on Dec. 20, 2018, now abandoned.
(60) Provisional application No. 62/782,943, filed on Dec. 20, 2018.

(51) Int. Cl. A61K 9/00; A61K 31/485; A61K 47/02; A61K 47/18; A61K 47/26; A61P 25/32; A61P 25/36

(52) U.S. Cl. CPC A61K 9/0043; A61K 31/485; A61K 47/02; A61K 47/183; A61K 47/186; A61K 47/26; A61P 25/32; A61P 25/36

(57) ABSTRACT

Drug products adapted for nasal delivery, comprising a device filled with a pharmaceutical composition comprising naltrexone are provided. Formulations and methods of treating alcohol use disorder and related conditions with the drug products are also provided.

### CROSS REFERENCE TO RELATED APPLICATIONS

This application is a continuation of U.S. patent application Ser. No. 16/721,872, filed Dec. 19, 2019, which is a continuation-in-part of U.S. patent application Ser. No. 16/311,944, filed Dec. 20, 2018, and also claims the benefit of U.S. Provisional Application No. 62/782,943, filed Dec. 20, 2018, the entire disclosure of each of which is considered part of and are incorporated by reference in the disclosure of this application in their entireties. This application also incorporates by reference the disclosures of PCT Publication Nos. WO2017/223566 and WO2018/089709 as if written herein in their entireties.

### FIELD OF INVENTION

The invention relates generally to pharmaceutical compositions and more particularly to intranasal formulations comprising naltrexone and forms thereof, and methods of use thereof in the treatment of and conditions such as opioid overdose and symptoms thereof, and disorders such as alcohol use disorder including administering an intranasal formulation of the opioid antagonist naltrexone.

### BACKGROUND INFORMATION

Naltrexone was initially developed to treat opioid dependence due to its effect of blocking the euphoric effects of opioids. Naltrexone tablet formulations for oral administration have been used for treating opioid addiction since 1984. Long-acting depot forms of naltrexone to be administered once monthly or longer were developed to improve compliance. Data from clinical trials demonstrated that the depot formulations were effective in reducing relapse to opioid use. Currently, there is one intramuscular, extended-release formulation, and one oral formulation, of naltrexone (Vivitrol®) for monthly administration approved by the FDA. These formulations aim to maintain a relatively steady state of an amount of naltrexone sufficient to prevent naltrexone intoxication at all times.

Opioid overdose, a related but somewhat different problem, is a serious public health issue. In 2017, approximately 72,000 people died from drug overdoses. Most of these, around 49,000, involved opioids. Over 19,000 of these deaths involved prescription opioid analgesics other than non-methadone synthetics; almost 3,300 involved methadone; about 16,000 involved heroin; and almost 30,000 of these deaths were attributed to fentanyl and related synthetic opioids, a stroking increase over previous years. Taken together, the number of opioid-related overdose deaths in 2016 far exceeded both the peak number of H.I.V. related deaths and the peak number of fatalities related to firearms, and have drastically increased over the past nine years. A need remains for effective treatments to reverse opioid overdose.

Meanwhile, there also remains a need for treatments for “reward-based” disorders, often involving opioids or other abusable substances such as alcohol, but also involving other activities which stimulate the brain's centers of pleasure, reward, and reinforcement and lead to an unhealthy excess of consumption of those substances or engagement in those behaviors.

For example, alcohol can stimulate the brain's reward circuitry and can reinforce the continued drinking of alcohol. The problematic drinking of alcohol that becomes sufficiently severe is given the medical diagnosis of alcohol use disorder (AUD). Approximately 6.8 percent (16.3 million adults) in the United States over the age of 18 had an AUD in 2014. This includes 10.6 million men and 5.7 million women. In addition, in 2014, an estimated 679,000 adolescents between the ages 12-17 (2.7% of this age group) had an AUD. In 2012, 3.3 million deaths or 5.9 percent of all global deaths (7.6% for men and 4.0% for women) were attributable to alcohol consumption (WHO Global Status Report on Alcohol and Health, 2014).

To be diagnosed with an AUD in the United States, individuals must meet certain criteria outlined in the Diagnostic and Statistical Manual of Mental Disorders (DSM). For example, under the fifth edition of the DSM, any individual meeting two of the eleven criteria during the same 12-month period receives a diagnosis of AUD. The severity of an AUD-mild, moderate, or severe-is based on the number of criteria met. In Europe, individuals are screened using the Alcohol Use Disorders Identification Test (AUDIT). People with AUD drink to excess and, consequently, can endanger both themselves and others.

Alcohol abuse is a drinking pattern that results in significant and recurrent adverse consequences. Alcohol abusers may fail to fulfill major school, work, or family obligations. People with alcoholism (also known as alcohol dependence) have lost reliable control of their alcohol use and are often unable to stop drinking once they start. Alcohol dependence is characterized by tolerance (the need to drink more to achieve the same “high”) and withdrawal symptoms if drinking is suddenly stopped. Withdrawal symptoms may include nausea, sweating, restlessness, irritability, tremors, hallucinations and convulsions.

Problem drinking has multiple causes, with genetic, physiological, psychological, and social factors all playing a role. Not every individual is equally affected by each cause. For some with AUD, psychological traits such as impulsiveness, low self-esteem and a need for approval prompt inappropriate drinking. Genetic factors make some people especially vulnerable to alcohol dependence. AUD can cause physiological changes that make more drinking the only way to avoid discomfort and individuals with AUD may drink partly to reduce or avoid withdrawal symptoms.

People with disorders associated with reward-based behavior, such as AUD, can seek counseling and psychological therapy from health professionals including physicians, nutritionists, psychiatrists, psychologists, clinical social workers or by attending 12-step Alcoholic Anonymous meetings. However, for a variety of reasons, access to, acceptance of, and success of such resources can be limited.

Considerable resistance to the use of medications for the treatment of disorders associated with reward-based behavior, such as AUD, persists and current evidence shows that medications are underused in the treatment of AUD. In Europe, oral nalmefene has been approved, and can be taken by a patient while drinking. However, as of January 2015, disulfiram, acamprosate, and oral or extended release injectable naltrexone are the only drugs approved by the Food and Drug Administration in the United States specifically for the treatment of AUD. However, all of these medications must be taken in patients who can abstain from alcohol before the initiation of treatment or have completed alcohol withdrawal. Accordingly, there is still a need for medications which treat subjects with AUD who are still drinking alcohol.

Non-drug reward-based disorders manifest in similar psychological and behavioral patterns as substance use disorders. Specifically, craving, impaired control over the behavior, tolerance, withdrawal, and high rates of relapse can be seen in subjects who suffer from addictive behavior that has negative consequences to the person's physical, mental, social or financial well-being. (See, e.g., Marks, 1990; Lejoyeux et al, 2000; National Institute on Drug Abuse (NIDA) et al, 2002; Potenza, 2006; and Olsen, 2011). Drugs and non-drug rewards also demonstrate similar physiological manifestations. For example, functional neuroimaging studies in humans have shown that gambling (Breiter et al, 2001), shopping (Knutson et al, 2007), orgasm (Komisaruk et al, 2004), playing video games (Koepp et al, 1998; Hoeft et al, 2008) and the sight of appetizing food (Wang et al, 2004) activate many of the same brain regions (i.e., the mesocorticolimbic system and extended amygdala) as drugs of abuse (Volkow et al, 2004).

An intranasal (IN) formulation of naltrexone has the potential to be used for reversing opioid overdose, and for treating reward-based disorders without the use of needles or an extended-release formulation. While studies have shown that opioid antagonists, such as naltrexone, administered in oral or injectable forms, can reverse opioid overdose, and can decrease alcohol drinking and operant responding for it, there remains a substantial need for a simple, fast and compliant means of treating such conditions.

### SUMMARY

Disclosed herein are intranasal formulations comprising an aqueous solution comprising naltrexone or a pharmaceutically acceptable salt thereof.

Also disclosed herein are methods of treatment of opioid overdose or a reward-based disorder such as alcohol use disorder (AUD) in a subject comprising administering to the subject an IN formulation comprising a therapeutically effective amount of naltrexone or a pharmaceutically acceptable salt thereof.

Also disclosed herein is a device adapted for nasal delivery of a pharmaceutical composition to a subject experiencing opioid overdose or having a reward-based disorder such as alcohol use disorder (AUD), comprising a therapeutically effective amount of a pharmaceutical formulation as disclosed herein. In certain embodiments, the device is pre-primed. In certain embodiments, the device can be primed before use. In certain embodiments, the device is a single-dose device. In certain embodiments, the device is a multi-dose device.

### BRIEF DESCRIPTION OF THE DRAWINGS

FIG. 1 shows the mean naltrexone plasma concentration versus time profiles following the administration of Test Product 1, Test Product 2, Test Product 3 and Reference Product, in linear scale out to 12 hours.

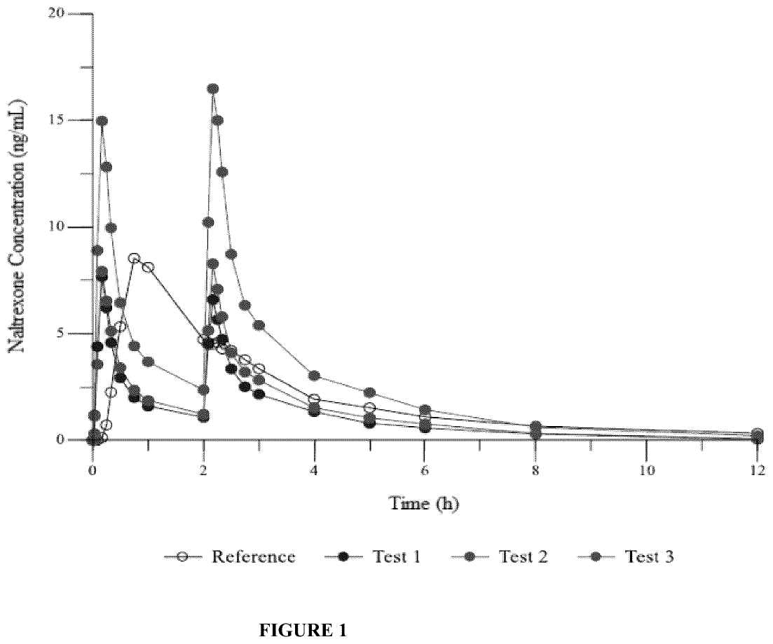

FIG. 2 shows the mean natlrexone plasma concentration versus time profiles following the administration of Test Product 1, Test Product 2, Test Product 3 and Reference Product, in semi-logarithmic scale out to 12 hours.

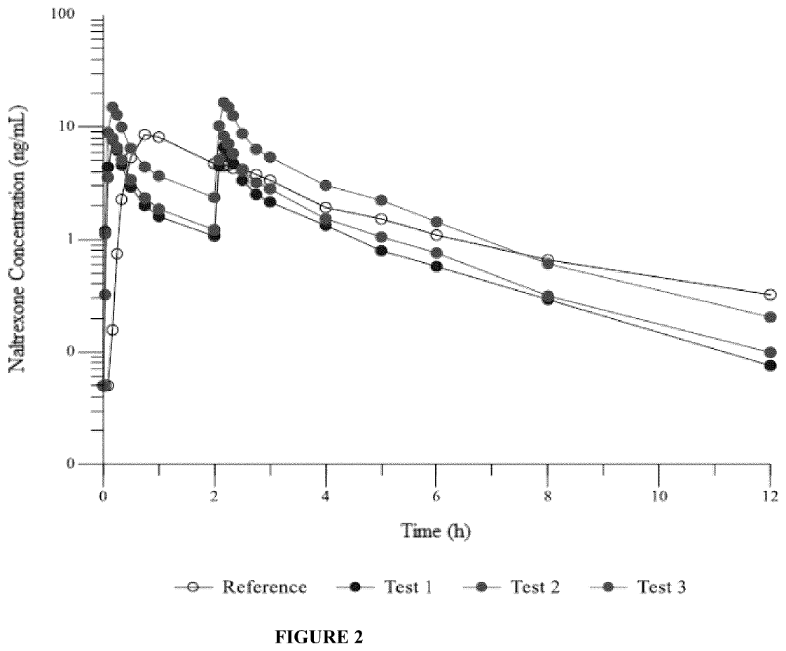

FIG. 3 shows the mean 6β-naltrexol (the main naltrexone metabolite) plasma concentration versus time (truncated at 12 h) profiles following the administration of Test Product 1, Test Product 2, Test Product 3 and Reference Product, in linear scale out to 12 hours.

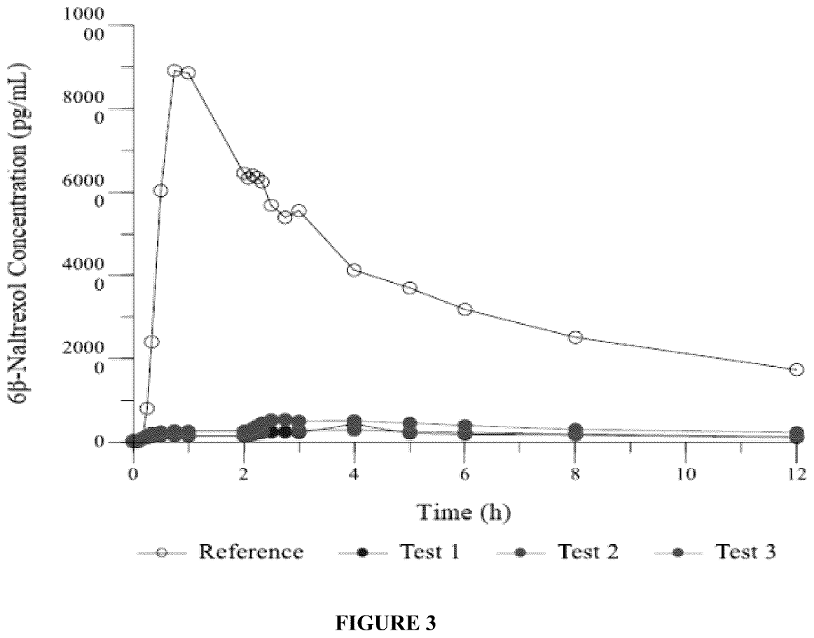

FIG. 4 shows the mean 6β-naltrexol (the main naltrexone metabolite) plasma concentration versus time (truncated at 12 h) profiles following the administration of Test Product 1, Test Product 2, Test Product 3 and Reference Product, in semi-logarithmic scale out to 12 hours.

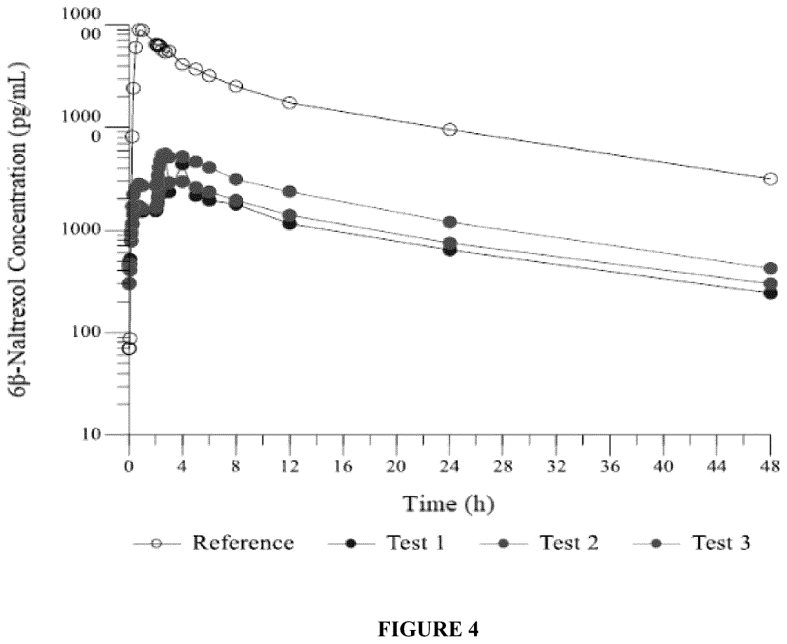

### DETAILED DESCRIPTION

The following embodiments further illustrate the invention disclosed herein.

Provided herein is Embodiment 1: an intranasal formulation comprising an aqueous solution comprising between about 1 mg and about 4 mg naltrexone, or a pharmaceutically acceptable salt thereof.

Also provided herein is Embodiment 2: an intranasal formulation comprising, in a volume of about 50 to about 250 μL (preferably about 50 to about 150 μL), an aqueous solution comprising between about 10 mg/mL and about 40 mg/mL (preferably about 10 mg/mL and about 30 mg/mL) naltrexone, or a pharmaceutically acceptable salt thereof.

The disclosure further provides the following embodiments.

Embodiment 3: The formulation as recited in either Embodiment 1 or Embodiment 2, additionally comprising:
an isotonicity agent;
a preservative;
a stabilizing agent;
an absorption enhancer; and
an amount of water sufficient to achieve a final volume of about 50 to about 250 μL (preferably about 50 to about 150 μL).

Embodiment 4: The formulation as recited in Embodiment 3, comprising:
between about 1 mg and about 3 mg naltrexone or a pharmaceutically acceptable salt thereof;
between about 0.1 mg and about 1.2 mg of the isotonicity agent;
between about 0.001 mg and about 0.1 mg of the preservative;
between about 0.1 mg and about 0.5 mg of the stabilizing agent;
between about 0.05 mg and about 2.5 mg of the absorption enhancer; and
an amount of water sufficient to achieve a final volume of about 50 to about 250 μL (preferably about 50 to about 150 μL).

Embodiment 5: The formulation as recited in Embodiment 3, comprising:
between about 1% and about 3% naltrexone or a pharmaceutically acceptable salt thereof;
between about 0.1% and about 1.2% of the isotonicity agent;
between about 0.001% and about 0.1% of the preservative;
between about 0.1% and about 0.5% of the stabilizing agent;
between about 0.05% mg and about 2.5% of the absorption enhancer.

Embodiment 6: The formulation as recited in either Embodiment 4 or Embodiment 5, wherein:
the isotonicity agent is NaCl;
the preservative is benzalkonium chloride;
the stabilizing agent is disodium edetate; and
the absorption enhancer is an alkylsaccharide.

Embodiment 7: The formulation as recited in Embodiment 6, wherein the alkylsaccharide is dodecyl maltoside.

Embodiment 8: The formulation as recited in Embodiment 7, comprising:
between about 1 mg and about 3 mg naltrexone or a pharmaceutically acceptable salt thereof;
between about 0.1 mg and about 1.2 mg of NaCl;
between about 0.001 mg and about 0.1 mg of benzalkonium chloride;
between about 0.15 mg and about 0.5 mg of disodium edetate;
between about 0.05 mg and about 2.5 mg of dodecyl maltoside; and
an amount of water sufficient to achieve a final volume of about 50 to about 250 ∥L (preferably about 50 to about 150 μL).

Embodiment 9: The formulation as recited in Embodiment 8, comprising between about 0.1 mg to about 0.5 mg of dodecyl maltoside.

Embodiment 10: The formulation as recited in Embodiment 9, comprising about 0.25 mg of dodecyl maltoside.

Embodiment 11: The formulation as recited in Embodiment 8, comprising about 0.2 mg and about 0.3 mg of disodium edetate.

Embodiment 12: The formulation as recited in Embodiment 9, comprising:
between about 1 mg and about 3 mg naltrexone or a pharmaceutically acceptable salt thereof;
between about 0.3 mg and about 0.7 mg of NaCl;
about 0.02 mg of benzalkonium chloride;
about 0.3 mg of disodium edetate;
about 0.25 mg of dodecyl maltoside; and an amount of water sufficient to achieve a final volume of about 50 to about 250 μL (preferably about 50 to about 150 μL).

Embodiment 13: The formulation as recited in Embodiment 10, wherein the amount of water is sufficient to achieve a final volume of about 80 to about 120 μL.

Embodiment 14: The formulation as recited in Embodiment 11, wherein the amount of water is sufficient to achieve a final volume of about 100 μL.

Embodiment 15: The formulation as recited in Embodiment 7, comprising:
between about 1% and about 3% naltrexone or a pharmaceutically acceptable salt thereof;
between about 0.1% and about 1.2% of NaCl;
between about 0.001% and about 0.1% of benzalkonium chloride;
between about 0.15% and about 0.5% of disodium edetate;
between about 0.05% and about 2.5% of dodecyl maltoside; and
water.

Embodiment 16: The formulation as recited in Embodiment 15, comprising between about 0.1% to about 0.5% of dodecyl maltoside.

Embodiment 17: The formulation as recited in Embodiment 16, comprising about 0.25% of dodecyl maltoside.

Embodiment 18: The formulation as recited in Embodiment 15, comprising about 0.2% and about 0.3% of disodium edetate.

Embodiment 19: The formulation as recited in Embodiment 17, comprising:
between about 1% and about 3% naltrexone or a pharmaceutically acceptable salt thereof;
between about 0.3% and about 0.7% of NaCl;
about 0.02% of benzalkonium chloride;
about 0.3% of disodium edetate;
about 0.25% of dodecyl maltoside; and
water.

Embodiment 20: The formulation as recited in Embodiment 19, wherein the amount of water is sufficient to achieve a final volume of about 50 to about 150 μL.

Embodiment 21: The formulation as recited in Embodiment 20, wherein the amount of water is sufficient to achieve a final volume of about 100 μL.

Embodiment 22: The formulation as recited in any of Embodiments 1-21, wherein the naltrexone is naltrexone hydrochloride.

Embodiment 23: The formulation as recited in Embodiment 22, comprising about 1.2 mg, about 1.6 mg, about 2.0 mg, or about 3.0 mg naltrexone or an equivalent amount of naltrexone hydrochloride.

Also provided herein is Embodiment 24: a method of treatment of opioid overdose or a reward-based disorder in a subject, comprising administering to the subject an intranasal formulation comprising an aqueous solution comprising between about 1 mg and about 4 mg naltrexone or a pharmaceutically acceptable salt thereof.

Also provided herein is Embodiment 25: a method of treatment of opioid overdose or a reward-based disorder in a subject, comprising administering to the subject a first intranasal formulation comprising an aqueous solution comprising between about 1 mg and about 4 mg naltrexone or a pharmaceutically acceptable salt thereof and administrating a second intranasal formulation comprising an aqueous solution comprising between about 1 mg and about 4 mg naltrexone or a pharmaceutically acceptable salt thereof.

Also provided herein is Embodiment 26: a method of treatment of opioid overdose or a reward-based disorder in a subject, comprising administering to the subject an intranasal formulation comprising, in a volume of about 50 to about 250 μL (preferably about 50 to about 150 μL), an aqueous solution comprising between about 10 mg/mL and about 40 mg/mL (preferably about 10 mg/mL and about 30 mg/mL) naltrexone or a pharmaceutically acceptable salt thereof.

Also provided herein is Embodiment 27: a method treatment of opioid overdose or a reward-based disorder in a subject, comprising administering to the subject a first intranasal formulation comprising, in a volume of about 50 to about 250 ul, an aqueous solution comprising between about 10 mg/mL and about 40 mg/mL naltrexone or a pharmaceutically acceptable salt thereof and administrating a second intranasal formulation comprising, in a volume or about 50 to about 250 μL, an aqueous solution comprising between about 10 mg/ml and about 40 mg/mL naltrexone or a pharmaceutically acceptable salt thereof.

Embodiment 28: The method as recited in either Embodiment 25 or claim Embodiment 27 wherein the second intranasal formulation is administered between about 1 hour and about 3 hours after the administration of the first intranasal formulation.

Embodiment 29: The method as recited in any of Embodiments 24-28, wherein the intranasal formulation additionally comprises:
an isotonicity agent;
a preservative;
a stabilizing agent;
an absorption enhancer; and
an amount of water sufficient to achieve a final volume of about 50 to about 250 μL (preferably about 50 to about 150 μL).

Embodiment 30: The method as recited in Embodiment 29, wherein the intranasal formulation comprises:
between about 1 mg and about 3 mg naltrexone or a pharmaceutically acceptable salt thereof;
between about 0.1 mg and about 1.2 mg of the isotonicity agent;
between about 0.001 mg and about 0.1 mg of the preservative;
between about 0.1 mg and about 0.5 mg of the stabilizing agent;
between about 0.05 mg and about 2.5 mg of the absorption enhancer; and
an amount of water sufficient to achieve a final volume of about 50 to about 250 μL (preferably about 50 to about 150 μL).

Embodiment 31: The method as recited in Embodiment 29, wherein the intranasal formulation comprises:
between about 1% and about 3% naltrexone or a pharmaceutically acceptable salt thereof;
between about 0.1% and about 1.2% of the isotonicity agent;
between about 0.001% and about 0.1% of the preservative;
between about 0.1% and about 0.5% of the stabilizing agent;
between about 0.05% mg and about 2.5% of the absorption enhancer.

Embodiment 32: The method as recited in either Embodiment 30 or Embodiment 28, wherein:
the isotonicity agent is NaCl;
the preservative is benzalkonium chloride;
the stabilizing agent is disodium edetate; and
the absorption enhancer is an alkylsaccharide.

Embodiment 33: The method as recited in Embodiment 32, wherein the alkylsaccharide is dodecyl maltoside.

Embodiment 34: The method as recited in Embodiment 33, wherein the intranasal formulation comprises:
between about 1 mg and about 3 mg naltrexone or a pharmaceutically acceptable salt thereof;
between about 0.1 mg and about 1.2 mg of NaCl;
between about 0.001 mg and about 0.1 mg of benzalkonium chloride;
between about 0.15 mg and about 0.5 mg of disodium edetate;
between about 0.05 mg and about 2.5 mg of dodecyl maltoside; and
an amount of water sufficient to achieve a final volume of about 50 to about 250 μL (preferably about 50 to about 150 μL).

Embodiment 35: The method as recited in Embodiment 34, wherein the intranasal formulation comprises between about 0.1 mg to about 0.5 mg of dodecyl maltoside.

Embodiment 36: The method as recited in Embodiment 35, wherein the intranasal formulation comprises about 0.25 mg of dodecyl maltoside.

Embodiment 37: The method as recited in Embodiment 34, wherein the intranasal formulation comprises between about 0.2 mg and about 0.3 mg of disodium edetate.

Embodiment 38: The method as recited in Embodiment 36, wherein the intranasal formulation comprises:
between about 1 mg and about 3 mg naltrexone or a pharmaceutically acceptable salt thereof;
between about 0.3 mg and about 0.7 mg of NaCl;
about 0.02 mg of benzalkonium chloride;
about 0.3 mg of disodium edetate;
about 0.25 mg of dodecyl maltoside; and
an amount of water sufficient to achieve a final volume of about 50 to about 250 μL (preferably about 50 to about 150 μL).

Embodiment 39: The method as recited in Embodiment 38, wherein the amount of water is sufficient to achieve a final volume of about 50 to about 150 μL.

Embodiment 40: The method as recited in Embodiment 39, wherein the amount of water is sufficient to achieve a final volume of about 100 μL.

Embodiment 41: The method as recited in Embodiment 33, comprising:
between about 1% and about 3% naltrexone or a pharmaceutically acceptable salt thereof;
between about 0.1% and about 1.2% of NaCl;
between about 0.001% and about 0.1% of benzalkonium chloride;
between about 0.1% and about 0.5% of disodium edetate;
between about 0.05% and about 2.5% of dodecyl maltoside; and water.

Embodiment 42: The method as recited in Embodiment 41, wherein the intranasal formulation comprises between about 0.1% to about 0.5% of dodecyl maltoside.

Embodiment 43: The method as recited in Embodiment 42, wherein the intranasal formulation comprises about 0.25% of dodecyl maltoside.

Embodiment 44: The method as recited in Embodiment 41, wherein the intranasal formulation comprises between about 0.2% and about 0.3% of disodium edetate.

Embodiment 45: The method as recited in Embodiment 43, wherein the intranasal formulation comprises:
between about 1% and about 3% naltrexone or a pharmaceutically acceptable salt thereof;
between about 0.3% and about 0.7% of NaCl;
about 0.02% of benzalkonium chloride;
about 0.3% of disodium edetate;
about 0.25% of dodecyl maltoside; and
water.

Embodiment 46: The method as recited in Embodiment 45, wherein the amount of water is sufficient to achieve a final volume of about 50 to about 150 μL.

Embodiment 47: The method as recited in Embodiment 46, wherein the amount of water is sufficient to achieve a final volume of about 100 μL.

Embodiment 48: The method as recited in any of Embodiments 24-47, wherein the naltrexone is naltrexone hydrochloride.

Embodiment 49: The method as recited in Embodiment 48, wherein the intranasal formulation comprises about 1.2 mg, about 1.6 mg, about 2.0 mg, or about 3.0 mg naltrexone or an equivalent amount of naltrexone hydrochloride.

Embodiment 50: The method as recited in any of Embodiments 24-49, wherein the method treats opioid overdose.

Embodiment 51: The method as recited in any of Embodiments 24-49, wherein the method treats a reward-based disorder.

Embodiment 52: The method as recited in Embodiment 48, wherein the reward-based disorder is a substance use disorder.

Embodiment 53: The method as recited in Embodiment 48, wherein the substance use disorder is alcohol use disorder.

Embodiment 54: The method as recited in Embodiment 53, wherein the intranasal formulation comprising naltrexone is administered prior to ingestion of alcohol.

Embodiment 55: The method as recited in Embodiment 54, wherein the intranasal formulation comprising naltrexone is administered about 1-2 hours prior to ingestion of alcohol.

Embodiment 56: The method as recited in Embodiment 54, wherein the intranasal formulation comprising naltrexone is administered about 0.5 to about 1 hours prior to ingestion of alcohol.

Embodiment 57: The method as recited in Embodiment 54, wherein the intranasal formulation comprising naltrexone is administered about 10 to about 30 minutes prior to ingestion of alcohol.

Embodiment 58: The method as recited in Embodiment 54, wherein the intranasal formulation comprising naltrexone is administered about 5 to about 10 minutes prior to ingestion of alcohol.

Embodiment 59: The method as recited in Embodiment 53, wherein the intranasal formulation comprising naltrexone is administered contemporaneously with the ingestion of alcohol.

Embodiment 57: The method as recited in Embodiment 53, wherein the intranasal formulation comprising naltrexone is administered within 0.5 hours after commencement of ingestion of alcohol.

Embodiment 58: The method as recited in any of Embodiments 24-57, wherein the intranasal formulation comprising naltrexone is administered to a subject from once to four times per day.

Embodiment 59: The method as recited in Embodiment 58, wherein the intranasal formulation comprising naltrexone is administered in doses of about 1.2 mg, about 1.6 mg, about 2 mg, or about 3 mg throughout the day as needed by the subject.

Embodiment 60: The method as recited in Embodiment 58, wherein the intranasal formulation comprising naltrexone is administered as a first dose of about 1.2 mg, about 1.6 mg, about 2 mg, or about 3 mg in the morning, and subsequent doses of about 1.2 mg, about 1.6 mg, about 2 mg, or about 3 mg as needed prior to consumption of alcohol.

Also provided herein is Embodiment 61: A multi-dose device adapted for nasal delivery of a pharmaceutical formulation to a subject experiencing an opioid overdose or having a reward-based disorder, comprising a plurality of doses each comprising an intranasal formulation as recited in any of Embodiments 1-23.

Embodiment 62: The device as recited in Embodiment 61, wherein about 0.05 to about 0.2 mL (preferably about 0.05 to about 0.15 mL) of said formulation is delivered to the subject in each dose.

Embodiment 63: The device as recited in Embodiment 62, wherein about 0.1 mL of said formulation is delivered to the subject in each dose.

Also provided herein is Embodiment 64: The formulation, method, or device as recited in any one of the preceding Embodiments, wherein:
where “comprising between about 1 mg and about 4 mg naltrexone, or a pharmaceutically acceptable salt thereof”, the amount comprised is about 3 mg naltrexone, or a pharmaceutically acceptable salt thereof; or
where the method recites “comprising between about 10 mg/mL and about 40 mg/mL (preferably about 10 mg/mL and about 30 mg/mL) naltrexone, or a pharmaceutically acceptable salt thereof”, the concentration comprised is about 30 mg/mL mg naltrexone, or a pharmaceutically acceptable salt thereof.

Embodiment 64: Also provided is a formulation chosen from those described in the Examples disclosed herein.

Also provided are embodiments wherein any embodiment above may be combined with any one or more of these embodiments, provided the combination is not mutually exclusive.

### Definitions

As use herein, the following terms have the meanings indicated.

When ranges of values are disclosed, unless otherwise specified, this notation is intended to include the numbers themselves and the range between them. This range may be integral or continuous between and including the end values. By way of example, the range “from 2 to 6 doses” is intended to include two, three, four, five, and six doses, since doses come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

The term “absorption enhancer,” as used herein, refers to a functional excipient included in formulations to improve the absorption of a pharmacologically active drug. This term usually refers to an agent whose function is to increase absorption by enhancing membrane permeation, rather than increasing solubility. As such, such agents are sometimes called permeation enhancers. Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, dodecyl maltoside, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, sodium lauryl sulfate, lysophosphatidylcholine, menthol, poloxamer 407 or F68, poly-L-arginine, polyoxyethylene-9-lauryl ether, polysorbate 80, propylene glycol, quillaja saponin, salicylic acid, sodium salt, β-sitosterol-β-D-glucoside, sucrose cocoate, taurocholic acid, taurodeoxycholic acid, taurodihydrofusidic acid, and tetradecyl maltoside. Alkylsaccharides (e.g., nonionic alkylsaccharide surfactants such as alkylglycosides and sucrose esters of fatty acids that consist of an aliphatic hydrocarbon chain coupled to a sugar moiety by a glycosidic or ester bond, respectively), cyclodextrins (cyclic oligosaccharides composed of six or more monosaccharide units with a central cavity, which form inclusion complexes with hydrophobic molecules and they have primarily been used to increase drug solubility and dissolution and to enhance low molecular weight drug absorption), chitosans (linear cationic polysaccharides produced from the deacetylation of chitin), and bile salts and their derivatives (such as sodium glycocholate, sodium taurocholate, and sodium taurodihydrofusidate) tend to be amongst the best-tolerated absorption enhancers. See, e.g., Aungst, AAPS Journal 14 (1): 10-8, 2011; Maggio, J. Excipients and Food Chem. 5 (2): 100-12, 2014.

The term “addiction,” as used herein, refers to a medical condition characterized by compulsive engagement in rewarding stimuli despite adverse consequences. The term, “addictive behavior,” as used herein, refers to a behavior that is both rewarding and reinforcing.

The term “agonist,” as used herein, refers to a moiety that interacts with and activates a receptor, and thereby initiates a physiological or pharmacological response characteristic of that receptor. The term “antagonist,” as used herein, refers to a moiety that competitively binds to a receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

The term “alcohol use disorder” is defined by criteria set forth the Diagnostic and Statistical Manual of Mental Disorders (DSM, most recent revision, presently DSM-V) in the US, or by similar criteria set forth in corresponding well-accepted standards such as the World Health Organization's ICD (International Statistical Classification of Diseases and Related Health Problems, most recent revision, presently the ICD-10). Related terms and disorders include “alcohol abuse” and “alcohol dependence” (used in DSM-IV), “alcohol harmful use” and “alcohol dependence syndrome” (used in the ICD-10), and alcoholism.

The term “antimicrobial preservative,” as used herein, refers to a pharmaceutically acceptable excipient with antimicrobial properties which is added to a pharmaceutical composition to maintain microbiological stability. Compounds act both as preservatives and stabilizers.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “pharmaceutical composition” is used herein interchangeably with the term “Pharmaceutical formulation,” or just “formulation,” and denotes an active pharmaceutical ingredient (i.e., a drug substance) in combination with at least one pharmaceutically acceptable excipient or carrier.

The term “equivalent,” as used herein refers to a weight of the opioid antagonist naltrexone and pharmaceutically acceptable salts thereof that is equimolar to a specified weight of naltrexone hydrochloride.

The term “excipient,” as used herein refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The term “therapeutically effective dose,” as used herein refers to a dose that is effective to treat a disease, to decrease one or more observable symptoms of a disease, or to delay onset or progression of or mitigate the symptoms of a more serious condition that often follows after the condition that a patient is currently experiencing. A therapeutically effective dose may, but need not necessarily, completely eliminate all symptoms of the disease.

The term “in need of treatment” and the term “in need thereof” when referring to treatment are used interchangeably and refer to a judgment made by a caregiver (e.g. physician, nurse, nurse practitioner, that a subject will benefit from treatment.

As used herein, two embodiments are “mutually exclusive” when one is defined to be something which is different than the other. For example, an embodiment wherein the amount of naltrexone hydrochloride is specified to be 3 mg is mutually exclusive with an embodiment wherein the amount of naltrexone hydrochloride is specified to be 2 mg. However, an embodiment wherein the amount of naltrexone hydrochloride is specified to be 4 mg is not mutually exclusive with an embodiment in which less than about 10% of said pharmaceutical composition leaves the nasal cavity via drainage into the nasopharynx or externally.

The term “naloxone,” as used herein, refers to a compound of the following structure:

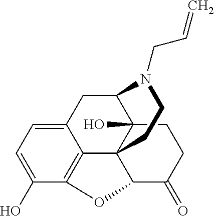

or a pharmaceutically acceptable salt, hydrate, or solvate thereof. The CAS registry number for naloxone is 465-65-6. Other names for naloxone include: 17-allyl-4,5a-epoxy-3,14-dihydroxymorphinan-6-one; (−)-17-allyl-4,5α-epoxy-3,14-dihydroxymorphinan-6-one; 4,5a-epoxy-3,14-dihydroxy-17-(2-propenyl) morphinan-6-one; and (−)-12-allyl-7,7a,8,9-tetrahydro-3,7a-dihydroxy-4aH-8,9c-iminoethanophenanthro [4,5-bcd]furan-5 (6H)-one. Naloxone hydrochloride may be anhydrous (CAS Reg. No. 357-08-4) and also forms a dihydrate (CAS No. 51481-60-8). It has been sold under various brand names including Narcan®, Nalone®, Nalossone®, Naloxona®, Naloxonum®, Narcanti®, and Narcon®.

The term “naltrexone,” as used herein, refers to a compound of the following structure:

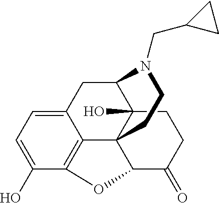

or a pharmaceutically acceptable salt, hydrate, or solvate thereof. The CAS registry number for naltrexone is 16590-41-3. Other names for naltrexone include: 17-(cyclopropylmethyl)-4,5α-epoxy-3,14-dihydroxymorphinan-6-one; (5α)-17-(cyclopropylmethyl)-3,14-dihydroxy-4,5-epoxymorphinan-6-one; and (1S,5R,13R,17S)-4-(cyclopropylmethyl)-10,17-dihydroxy-12-oxa-4-azapentacyclo [9.6.1.01, 13.05, 17.07, 18]octadeca-7 (18), 8, 10-trien-14-one. The term “naltrexone” includes “naltrexone hydrochloride.” Naltrexone hydrochloride (CAS Reg. No. 16676-29-2) has been marketed under the trade names Antaxone®, Depade®, Nalorex®, Revia®, Trexan®, Vivitrex®, and Vivitrol®.

The term “methylnaltrexone,” as used herein, refers to a pharmaceutically acceptable salt comprising the cation (5α)-17-(cyclopropylmethyl)-3,14-dihydroxy-17-methyl-4,5-epoxymorphinanium-17-ium-6-one a compound of the following structure:

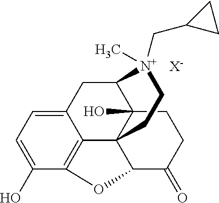

wherein X⁻ is a pharmaceutically acceptable anion. Methylnaltrexone bromide (CAS Reg. No. 75232-52-7) has been marketed under the trade name Relistor®.

The term “nalmefene,” as used herein, refers to 17-cyclopropylmethyl-4,5α-epoxy-6-methylenemorphinan-3,14-diol, a compound of the following structure:

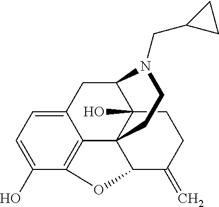

Nalmefene hydrochloride (CAS Reg. No. 58895-64-0) has been marketed under the trade names Nalmetrene®, Cervene®, Revex®, Arthrene®, and Incystene®.

The term “nostril,” as used herein, is synonymous with “naris.”

The term “opioid antagonist” includes, in addition to naltrexone and pharmaceutically acceptable salts thereof: naloxone, methylnaltrexone, and nalmefene, and pharmaceutically acceptable salts thereof. In certain embodiments, the opioid antagonist is naltrexone hydrochloride. In certain embodiments, the opioid antagonist is naloxone. In certain embodiments, the opioid antagonist is methylnaltrexone bromide. In certain embodiments, the nasally administering is accomplished using a device described herein.

The term “pharmaceutical composition,” as used herein, refers to a composition comprising at least one active ingredient; including but not limited to, salts, solvates and hydrates of the opioid antagonist naltrexone, whereby the composition is amenable to use for a specified, efficacious outcome in a mammal (for example, without limitation, a human).

The term “reinforcing stimuli,” as used herein refers to stimuli that increase the probability of repeating behaviors paired with them.

The term, “rewarding stimuli,” as used herein, refers to stimuli that the brain of a subject interprets as intrinsically positive or as something to be approached. A rewarding stimulus typically results in the release of dopamine in the brain of the subject.

As used herein, a “reward-based disorder” is a disorder associated with reward-based behavior, wherein the user pursues engagement in and/or engages in rewarding stimuli despite adverse consequences. Reward-based disorders include substance use disorders such as alcohol use disorder, as well as addictions, as well as disorders of activity engagement such as gambling disorder (compulsive gambling), eating disorders (binge eating, bulimia nervosa), kleptomania, pyromania, and the like.

The term “subject,” as used herein, is intended to be synonymous with “patient,” and refers to any mammal (preferably human) afflicted with a condition likely to benefit from a treatment with a therapeutically effective amount of the opioid antagonist naltrexone or a salt thereof.

The term “substance use disorder” is defined by criteria set forth the Diagnostic and Statistical Manual of Mental Disorders (DSM, most recent revision, presently DSM-V) in the US, or by similar criteria set forth in corresponding well-accepted standards such as the World Health Organization's ICD (International Statistical Classification of Diseases and Related Health Problems, most recent revision, presently the ICD-10). Related terms and disorders include “substance abuse” and “substance dependence” (used in DSM-IV). Substance use disorders occur when the recurrent use of alcohol and/or drugs causes clinically and functionally significant impairment, such as health problems, disability, and failure to meet major responsibilities at work, school, or home. According to the DSM-5, a diagnosis of substance use disorder is based on evidence of impaired control, social impairment, risky use, and pharmacological criteria. Substances which may be the focus of a substance use disorder include abusable substances such as opioids, alcohol, tobacco, cannabinoids, stimulants such as cocaine and amphetamines, depressants such as benzodiazepines, hallucinogens, inhalants, and the like. Alcohol use disorder is a substance use disorder. Substance use disorders may be considered “reward-based disorders.”

The term “tonicity agent,” as used herein, refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

As used herein, “treating,” “treatment,” and the like means ameliorating a disorder, so as to reduce or eliminate its cause, its progression, its severity, or one or more of its symptoms, or otherwise beneficially alter the disease in a subject.

As used herein, the term “AUC” refers to the area under the drug plasma concentration-time curve. As used herein, the term “AUC₀₋ₜ” refers to the area under the drug plasma concentration-time curve from t=0 to the last measurable concentration. As used herein, the term “AUC_{0-∞}” or “AUC_{0-inf}” refers to the area under the drug plasma concentration-time curve extrapolated to ∞ (infinity).

As used herein, the term “bioavailability (F)” refers to the fraction of a dose of drug that is absorbed from its site of administration and reaches, in an unchanged form, the systemic circulation. As used herein, the term “absolute bioavailability” is used when the fraction of absorbed drug is related to its IV bioavailability. It may be calculated using the following formula:

$$\\ F = \frac{{AUC}_{extravascular}}{{AUC}_{intravenous}} \times \frac{{Dose}_{intravenous}}{{Dose}_{extravascular}}$$

The term relative bioavailability (Fᵣₑₗ) is used to compare two different extravascular routes of drug administration and it may be calculated using the following formula:

$$\\ {F}_{rel} = \frac{{AUC}_{extravascular 1}}{{AUC}_{extravascular 2}} \times \frac{{Dose}_{extravascular 2}}{{Dose}_{extravascular 1}}$$

As used herein, the term “clearance (CL)” refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma. As used herein, the term “apparent clearance (CL/F)” refers to clearance that does not take into account the bioavailability of the drug. It is the ratio of the dose over the AUC.

As used herein, the term “Cₘₐₓ” refers to the maximum observed plasma concentration.

As used herein, the term “t_{1/2}” or “half-life” refers to the amount of time required for half of a drug to be eliminated from the body or the time required for a drug concentration to decline by half.

As used herein, the term “coefficient of variation (CV)” refers to the ratio of the sample standard deviation to the sample mean. It is often expressed as a percentage.

As used herein, the term “confidence interval” refers to a range of values which will include the true average value of a parameter a specified percentage of the time.

As used herein, the term “elimination rate constant (λ)” refers to the fractional rate of drug removal from the body. This rate is constant in first-order kinetics and is independent of drug concentration in the body. λ is the slope of the plasma concentration-time line (on a logarithmic y scale). The term “λ_{z},” as used herein, refers to the terminal phase elimination rate constant, wherein the “terminal phase” of the drug plasma concentration-time curve is a straight line when plotted on a semilogarithmic graph. The terminal phase is often called the “climination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

### Opioid Antagonists

Opioid receptor antagonists are a well-recognized class of chemical agents. They have been described in detail in the scientific and patent literature. Naltrexone and its active metabolite 6β-naltrexol are opioid antagonists, with no agonist properties, at the μ-opioid receptor (MOR), the κ-opioid receptor (KOR), and the δ-opioid receptor (DOR). Naltrexone operates by reversibly blocking the opioid receptors thereby attenuating the effects of opioids. Its mechanism of action in alcohol dependence is not fully understood but, without being limited by theory, naltrexone likely modulates the dopaminergic mesolimbic pathway (one of the primary centers for risk-reward analysis in the brain, and a tertiary pleasure center) which is believed to be a major center of the reward associated with addiction that all major drugs of abuse are believed to activate. The mechanism of action may be antagonism to endogenous opiates such as tetrahydropapaveroline, whose production is augmented in the presence of alcohol.

Naltrexone is commercially available as a hydrochloride salt. Naltrexone hydrochloride (17-(cyclopropylmethyl)-4,5α-epoxy-3,14-dihydroxymorphinan-6-one) is used to prevent euphorigenic effects in the treatment of patients addicted to opioids. It markedly blocks the physical dependence to intravenously administered opioids and motivates withdrawal from opioid dependency, but the patient does not develop tolerance or dependence to naltrexone. Naltrexone is also effective in reducing the craving for alcohol in the treatment of alcoholism, especially when combined with psychosocial therapy.

When naltrexone is administered intranasally, rather than orally, the bioavailability is significantly higher. When administered orally, despite being nearly completely absorbed from the gastrointestinal tract, naltrexone undergoes rapid and extensive first-pass metabolism to 6-β-naltrexol. As a result, the amount of naltrexone reaching systemic circulation is limited. The oral bioavailability of naltrexone has been reported to be as low as 5%. Gonzalez and Brogden, Drugs 35:192-213, 1988. Studies presented herein found the oral bioavailability of naltrexone to be similarly low, about 9%.

Provided herein are methods of treatment employing nasal delivery of a pharmaceutical composition to a patient, comprising a therapeutically effective amount of the opioid antagonist naltrexone. In certain embodiments, the therapeutically effective amount per dose is equivalent to about 1 to about 4 mg of naltrexone hydrochloride. In certain embodiments, the therapeutically effective amount is equivalent to about 1 to about 3 mg of naltrexone hydrochloride. In certain embodiments, the therapeutically effective amount per dose is equivalent to about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 mg of naltrexone hydrochloride. In certain embodiments, the therapeutically effective amount per dose is equivalent to about 1.2 mg of naltrexone hydrochloride. In certain embodiments, the therapeutically effective amount per dose is equivalent to about 1.6 mg of naltrexone hydrochloride. In certain embodiments, the therapeutically effective amount per dose is equivalent to about 2.0 mg of naltrexone hydrochloride. In certain embodiments, the therapeutically effective amount per dose is equivalent to about 3.0 mg of naltrexone hydrochloride. Multiple doses in succession may be taken to achieve therapeutic efficacy. In certain embodiments, the opioid antagonist is anhydrous naltrexone hydrochloride.

While many of the embodiments of the pharmaceutical compositions described herein will be described and exemplified with naltrexone, other opioid antagonists can be adapted for nasal delivery based on the teachings of the specification. In fact, it should be readily apparent to one of ordinary skill in the art from the teachings herein that the devices and pharmaceutical compositions described herein may be suitable for other opioid antagonists. The opioid receptor antagonists described herein include μ-opioid, κ-opioid, and δ-opioid receptor antagonists. Examples of useful opioid receptor antagonists include naltrexone, naloxone, methylnaltrexone, and nalmefene. Other useful opioid receptor antagonists are known in the art (e.g., U.S. Pat. No. 4,987,136).

### Pharmaceutical Formulations

Also provided are pharmaceutical compositions comprising the opioid antagonist naltrexone. In certain embodiments the pharmaceutical compositions comprise the opioid antagonist naltrexone and a pharmaceutically acceptable carrier. The carrier(s) must be “acceptable” in the sense of being compatible with the other ingredients of the formulation and not overly deleterious to the recipient thereof. Some embodiments of the present invention include a method of producing a pharmaceutical composition comprising admixing the opioid antagonist naltrexone and a pharmaceutically acceptable carrier. Pharmaceutical compositions are applied directly to the nasal cavity using the devices described herein. In the case of a spray, this may be achieved for example by means of a metering atomizing spray pump.

Liquid preparations include solutions, suspensions and emulsions, for example, water or water-propylene glycol solutions. Additional ingredients in liquid preparations may include: antimicrobial preservatives, such as benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof; and a suspending agent such as microcrystalline cellulose, carboxymethylcellulose sodium NF, polyacrylic acid, magnesium aluminum silicate, xanthan gum, and the like, and mixtures thereof.

Ideally, when an opioid antagonist is administered intranasally prior to ingestion of alcohol to treat AUD, the opioid antagonist is absorbed quickly, i.e., within about fifteen to about thirty minutes and/or yielding a time to the maximum plasma concentration (Tₘₐₓ) of about 25 to about 40 minutes. For example, in certain embodiments, the opioid antagonist is absorbed within the first 15 min after administration and the time to the maximum plasma concentration (Tₘₐₓ) is 25 min or less. Alternatively, the opioid antagonist is absorbed within the first 30 min after administration and the Tₘₐₓ is 40 min or less.

The use of absorption enhancers, such as alkylsaccharides (also referred to herein as alkylglycosides), cyclodextrins, and chitosans may increase the rate at which naltrexone is absorbed and decrease the Tₘₐₓ. Such absorption enhancers typically operate by affecting two primary mechanisms for nasal absorption: paracellular transport via opening of tight junctions between cells, and transcellular transport or transcytosis through cells via vesicle carriers.

In various aspects, alkylglycosides of the present invention may include, but not limited to: alkylglycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl-or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, one preferred range of alkyl chains is from about 9 to about 24 carbon atoms. An even more preferred range is from about 9 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, etc. These compositions are nontoxic, since they are degraded to an alcohol or fatty acid and an oligosaccharide, and amphipathic. Additionally, the linkage between the hydrophobic alkyl group and the hydrophilic saccharide can include, among other possibilities, a glycosidic, thioglycosidic, amide, ureide, or ester linkage.

As use herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. Accordingly, examples of saccharides include glucose, maltose, maltotriose, maltotetraose, sucrose and trehalose.

In one embodiment, an exemplary alkylsaccharide is an alkylmaltoside. Alkylmaltosides are glycosides of the disaccharide maltose and alcohols. Typical alkylmaltosides are dodecylmaltoside, tetradecylmaltoside and hexadecylmaltoside which consist of a 12, 14 and 16 carbon straight chain alcohol respectively, glycosidically attached to maltose. In an exemplary embodiment, the alkylglycoside is tetradecylmaltoside.

For example, one alkyl saccharide is 1-O-n-dodecyl-β-D-maltopyranoside (alternately referred to as lauryl-β-D-maltopyranoside, dodecyl maltopyranoside, dodecyl maltoside, Intravail®, and DDM; C₂₄H₄₆Q₁₁). Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Alkylsaccharides increase absorption by increasing paracellular permeability, as indicated by a decrease in transepithelial electrical resistance; they may also increase transcytosis. The effect is short-lived. Other alkylsaccharides include tetradecyl maltoside (TDM) and sucrose dodecanoate.

In sugar chemistry, an anomer is either of a pair of cyclic stereoisomers (designated α or β) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, α-D-glucopyranose and β-D-glucopyranose, the two cyclic forms of glucose, are anomers. Likewise, alkylglycosides occur as anomers. For example, dodecyl β-D-maltoside and dodecyl α-D-maltoside are two cyclic forms of dodecyl maltoside. The two different anomers are two distinct chemical structures, and thus have different physical and chemical properties. In one aspect of the invention, the alkylglycoside of the present invention is a β anomer. In an exemplary aspect, the alkylglycoside is a β anomer of an alkylmaltoside, such as tetradecyl-β-D-maltoside (TDM).

Thus, in one aspect of the present invention, the alkylglycoside used is a substantially pure alkylglycoside. As used herein a “substantially pure” alkylglycoside refers to one anomeric form of the alkylglycoside (either the α or β anomeric forms) with less than about 2% of the other anomeric form, preferably less than about 1.5% of the other anomeric form, and more preferably less than about 1% of the other anomeric form. In one aspect, a substantially pure alkylgycoside contains greater than 98% of either the α or β anomer. In another aspect, a substantially pure alkylgycoside contains greater than 99% of either the a or β anomer. In another aspect, a substantially pure alkylgycoside contains greater than 99.5% of either the α or β anomer. In another aspect, a substantially pure alkylgycoside contains greater than 99.9% of either the α or β anomer.

In certain embodiments, an intranasal formulation comprises about 0.001% to about 5.0% dodecyl maltoside by weight. In certain embodiments, an intranasal formulation comprises about 0.01% to about 2.5% dodecyl maltoside. In certain embodiments, an intranasal formulation comprises about 0.05% to about 2.5% dodecyl maltoside. In certain embodiments, an intranasal formulation comprises about 0.1% to about 0.5% dodecyl maltoside. In certain embodiments, an intranasal formulation comprises about 0.15% to about 0.35% dodecyl maltoside. In certain embodiments, an intranasal formulation comprises about 0.15% to about 0.2% dodecyl maltoside. In certain embodiments, an intranasal formulation comprises about 0.18% dodecyl maltoside. In certain embodiments, an intranasal formulation comprises about 0.2% to about 0.3% dodecyl maltoside. In certain embodiments, an intranasal formulation comprises about 0.25% dodecyl maltoside.

When 0.18% dodecyl maltoside was added to an intranasal formulation of sumatriptan, the maximum plasma concentration increased almost four-fold in comparison to Imitrex nasal spray and Tₘₐₓ was reduced from 1-2 hours to 8-10 minutes. Total exposure, as measured by the area under the concentration-time curve (AUC), increased 32%. An intranasal formulation of naltrexone has the potential to be used for treating AUD without the use of needles or an extended-release formulation. Inclusion of dodecyl maltoside may improve pharmacokinetic parameters in some applications.

Some absorption enhancing excipients can alter the paracellular and/or transcellular pathways, others can extend residence time in the nasal cavity or prevent metabolic changes. Without an absorption enhancer, the molecular-weight limit for nasal absorption is about 1 kDa, while administration of drugs in conjunction with absorption enhancers can enable the absorption of molecules from 1-30 kDa. Intranasal administration of most absorption enhancers, however, can cause nasal mucosa damage. Maggio, J. Excipients and Food Chem. 5 (2): 100-12, 2014.

Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, dodecyl maltoside, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, lauryl sulfate, lysophosphatidylcholine, menthol, poloxamer 407, poloxamer F68, poly-L-arginine, polyoxyethylene-9-lauryl ether, polysorbate 80, propylene glycol, quillaja saponin, salicylic acid, β-Sitosterol-β-D-glucoside, sucrose cocoate, taurocholic acid, taurodeoxycholic acid, taurodihydrofusidic acid, and tetradecyl maltoside.

The opioid antagonist naltrexone described herein can be formulated into pharmaceutical compositions using techniques well known to those in the art. Suitable pharmaceutically acceptable carriers, outside those mentioned herein, are known in the art.

The opioid antagonist naltrexone described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent. The opioid antagonist naltrexone described herein may form solvates with standard low molecular weight solvents using methods known to the skilled artisan.

Accordingly, provided herein are pharmaceutical formulations for intranasal administration comprising naltrexone or a salt thereof, e.g., naltrexone hydrochloride. In certain embodiments, the formulation is an aqueous solution. In certain embodiments, the formulation comprises, per dose, between about 25 and about 200 μL of the aqueous solution. In certain embodiments, the formulation comprises, per dose, between about 50 and about 200 μL of the aqueous solution. In certain embodiments, the formulation comprises, per dose, not more than about 140 μL. In certain embodiments, the formulation comprises, per dose, not more than about 100 μL.

In certain embodiments, the formulation comprises between about 1% (w/w) and about 4% (w/w) of naltrexone hydrochloride. In certain embodiments, the formulation comprises between about 1% (w/w) and about 3% (w/w) of naltrexone hydrochloride. In certain embodiments, the formulation comprises about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0% (w/w) of naltrexone hydrochloride. In certain embodiments, the formulation comprises about 1.2% (w/w) of naltrexone hydrochloride. In certain embodiments, the formulation comprises about 1.6% (w/w) of naltrexone hydrochloride. In certain embodiments, the formulation comprises about 2% (w/w) of naltrexone hydrochloride. In certain embodiments, the formulation comprises about 3% (w/w) of naltrexone hydrochloridc.

In certain embodiments, the formulation comprises between about 1 mg and about 4 mg of naltrexone hydrochloride. In certain embodiments, the formulation comprises between about 1 mg and about 3 mg of naltrexone hydrochloride. In certain embodiments, the formulation comprises between about 2 mg and about 4 mg of naltrexone hydrochloride. In certain embodiments, the formulation comprises about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 mg of naltrexone hydrochloride. In certain embodiments, the formulation comprises about 1.2 mg of naltrexone hydrochloride. In certain embodiments, the formulation comprises about 1.6 mg of naltrexone hydrochloride. In certain embodiments, the formulation comprises about 2 mg of naltrexone hydrochloride. In certain embodiments, the formulation comprises about 3 mg of naltrexone hydrochloride.

Aqueous formulations for intranasal administration disclosed herein may also include pharmaceutically acceptable excipients, such as one or more isotonicity agents, one or more preservatives, one or more stabilizing agents, one or more absorption enhancers, and one or more agents to adjust pH or buffer the solution.

In certain embodiments, the intranasal formulation additionally comprises am isotonicity agent, such as sodium chloride (NaCl).

In certain embodiments, the intranasal formulation additionally comprises a compound which is a preservative and/or surfactant.

In certain embodiments, the preservative and/or surfactant is chosen from benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof.

In certain embodiments, the intranasal formulation additionally comprises a stabilizing agent.

In certain embodiments, the stabilizing agent is disodium edetate (EDTA).

In certain embodiments, the pharmaceutical composition is in an aqueous solution of about 100 μL.

In certain embodiments, upon nasal delivery of said pharmaceutical composition to said patient, less than about 10% of said pharmaceutical composition leaves the nasal cavity via drainage into the nasopharynx or externally.

### Nasal Drug Delivery Devices and Kits

Also provided are pharmaceutical compositions in a device adapted for nasal delivery to a subject suffering AUD, comprising a therapeutically effective amount of the opioid antagonist naltrexone or pharmaceutically acceptable salt thereof. In certain embodiments, the device is pre-primed. In certain embodiments, the device can be primed before use. In certain embodiments, the device can be actuated with one hand.

Nasal delivery is considered an attractive route for systemic drug delivery, especially when rapid absorption and effect are desired. In addition, nasal delivery may help address issues related to unpleasant taste, poor bioavailability, slow absorption, drug degradation, adverse events (AEs) in the gastrointestinal tract, and avoids first-pass metabolism and the hepatic toxicity associated with long-term oral naltrexone usage.

Liquid nasal formulations are mainly aqueous solutions, but suspensions and emulsions can also be delivered.

Some emergency medical service (EMS) programs have developed a system using existing technologies of an approved drug and an existing medical device to administer the opioid antagonist naloxone intranasally, albeit in a non-FDA approved manner. This has been accomplished by using the injectable formulation (1 mg/mL) and administering 1 mL per nostril via a marketed nasal atomizer/nebulizer device. The system combines an FDA-approved naloxone injection product (with a Luer fitted tip, no needles) with a marketed, medical device called the Mucosal Atomization Device (MAD™ Nasal, Wolfe Tory Medical, Inc.). This initiative is consistent with the U.S. Needlestick Safety and Prevention Act (Public Law 106-430). The EMS programs recognize limitations of this system, one limitation being that it is not assembled and ready-to-use. Although this administration mode appears to be effective in reversing narcosis, the formulation is not concentrated for retention in the nasal cavity. The 1 mL delivery volume per nostril is larger than that generally utilized for intranasal drug administration. Therefore, there is loss of drug from the nasal cavity, due either to drainage into the nasopharynx or externally from the nasal cavity. The devices described herein are improved ready-to-use products specifically optimized, concentrated, and formulated for nasal delivery.

Metered spray pumps have dominated the nasal drug delivery market since they were introduced. The pumps typically deliver 100 μL (or other volumes in the range of 25-200 μL, and higher) per spray, and they offer high reproducibility of the emitted dose and plume geometry in in vitro tests.

Examples of standard metered spray pumps include those offered by Aptar Pharma, Inc., such as the multi-dose “classic technology platform” nasal spray devices. Such devices comprise a reservoir which holds multiple doses of the nasal spray formulation (e.g., 50, 100, 150, 200, 60, or 120 doses), a closure (e.g., screw, crimp, or snap-on), and an actuator which delivers anywhere from 45 to 1000 μL (e.g. 50, 100, 140, 150, or 200 μL) of fluid per actuation to comprise a single dose. The actuator may be configured to count doses, deliver gel formulations, deliver in an upside-down configuration, etc.

In traditional spray pump systems, antimicrobial preservatives are typically required to maintain microbiological stability in liquid formulations. However, preservative-free systems are also available, e.g. the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc.

The particle size and plume geometry can vary within certain limits and depend on the properties of the pump, the formulation, the orifice of the actuator, and the force applied. The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 μm. If the droplets are too large (>about 120 μm), deposition takes place mainly in the anterior parts of the nose, and if the droplets are too small (<about 10 μm), they can possibly be inhaled and reach the lungs, which should be avoided because of safety reasons. In its capacity as a surfactant, benzalkonium chloride can affect the surface tension of droplets from a delivered nasal spray plume, producing spherical or substantially spherical particles having a narrow droplet size distribution (DSD), as well as the viscosity of a liquid formulation.

Plume geometry, droplet size and DSD of the delivered plume subsequent to spraying may be measured under specified experimental and instrumental conditions by appropriate and validated and/or calibrated analytical procedures known in the art. These include photography, laser diffraction, and impaction systems (cascade impaction, NGI). Plume geometry, droplet size and DSD can affect pharmacokinetic outcomes such as Cₘₐₓ, Tₘₐₓ, and linear dose proportionality.

Droplet size distribution can be controlled in terms of ranges for the D10, D50, D90, span [(D90-D10)/D50], and percentage of droplets less than 10 mm. In certain embodiments, the formulation will have a narrow DSD. In certain embodiments, the formulation will have a D (v,50) of 30-70 μm and a D (v, 90) <100 μm.

In certain embodiments, the percent of droplets less than 10 μm will be less than 10%. In certain embodiments, the percent of droplets less than 10 μm will be less than 5%. In certain embodiments, the percent of droplets less than 10 μm will be less than 2%. In certain embodiments, the percent of droplets less than 10 μm will be less than 1%.

In certain embodiments, the formulation when dispensed by actuation from the device will produce a uniform circular plume with an ovality ratio close to 1. Ovality ratio is calculated as the quotient of the maximum diameter (Dₘₐₓ) and the minimum diameter (Dₘᵢₙ) of a spray pattern taken orthogonal to the direction of spray flow (e.g., from the “top”). In certain embodiments, the ovality ratio is less than ±2.0. In certain embodiments, the ovality ratio is less than ±1.5. In certain embodiments, the ovality ratio is less than ⊥1.3. In certain embodiments, the ovality ratio is less than ±1.2. In certain embodiments, the ovality ratio is less than ±1.1. In certain embodiments, the ovality ratio is about ±1.0.

The details and mechanical principles of particle generation for different types of nasal aerosol devices has been described. Reviewed in Vidgren and Kublik, Adv. Drug Deliv. Rev. 29:157-77, 1998. Traditional spray pumps replace the emitted liquid with air, and preservatives are therefore required to prevent contamination. However, driven by the studies suggesting possible negative effects of preservatives (e.g., irritation of nasal mucosa), pump manufacturers have developed different spray systems that avoid the need for preservatives. These systems use a collapsible bag, a movable piston, or a compressed gas to compensate for the emitted liquid volume (www.aptar.com and www.rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip (www.aptar.com). More recently, pumps have been designed with side-actuation and introduced for delivery of fluticasone furoate for the indication of seasonal and perennial allergic rhinitis. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (www.rexam.com and www.aptar.com).

Traditional, simple metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window, particularly if they are not used often. For expensive drugs and vaccines intended for single administration or sporadic use and where tight control of the dose and formulation is of particular importance, single-dose or bi-dose spray devices are preferred (www.aptar.com). A simple variant of a single-dose spray device (MAD™) is offered by LMA (LMA, Salt Lake City, UT, USA; www.lmana.com). A nosepicce with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, NC, USA; www.bdpharma.com) is used to deliver the influenza vaccine FluMist™ (www.flumist.com), approved for both adults and children in the US market. A similar device for two doses was marketed by a Swiss company for delivery of another influenza vaccine a decade ago.

Pre-primed single-and bi-dose devices are also available, and consist of a reservoir, a piston, and a swirl chamber (see, e.g., the UDS UnitDose and BDS BiDose™ devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (www.gsk.com) and Zomig® (www.az.com; Pfeiffer/Aptar single-dose device), the marketed influenza vaccine Flu-Mist (www.flumist.com; Becton Dickinson single-dose spray device), and the intranasal formulation of naloxone for opioid overdose rescue, Narcan Nasal® (narcan.com; Adapt Pharma) are delivered with this type of device.

In certain embodiments, the 90% confidence interval for dose delivered per actuation is ±about 2%. In certain embodiments, the 95% confidence interval for dose delivered per actuation is ±about 2.5%.

Historically, intranasal administration of drugs in large volume, such as from syringes adapted with mucosal atomizer devices, has encountered difficulty due to the tendency of some of the formulation to drip back out of the nostril or down the nasopharynx. Accordingly, in certain embodiments, upon nasal delivery of said pharmaceutical composition to said patient, less than about 20% of said pharmaceutical composition leaves the nasal cavity via drainage into the nasopharynx or externally. In certain embodiments, upon nasal delivery of said pharmaceutical composition to said patient, less than about 10% of said pharmaceutical composition leaves the nasal cavity via drainage into the nasopharynx or externally. In certain embodiments, upon nasal delivery of said pharmaceutical composition to said patient, less than about 5% of said pharmaceutical composition leaves the nasal cavity via drainage into the nasopharynx or externally.

Current container closure system designs for inhalation spray drug products include both pre-metered and device-metered presentations using mechanical or power assistance and/or energy from patient inspiration for production of the spray plume. Pre-metered presentations contain previously measured doses or a dose fraction in some type of units (e.g., single or multiple blisters or other cavities) that are subsequently inserted into the device during manufacture or by the patient before use. Typical device-metered units have a reservoir containing formulation sufficient for multiple doses that are delivered as metered sprays by the device itself when activated by the patient.

A new nasal drug delivery method, which can be adapted to any type of dispersion technology for both liquids and powders, is breath-powered Bi-Directional™ technology. This concept exploits natural functional aspects of the upper airways to offer a delivery method that may overcome many of the inherent limitations of traditional nasal devices. Breath-powered Bi-Directional™ devices consist of a mouthpiece and a sealing nosepiece with an optimized frusto-conical shape and comfortable surface that mechanically expands the first part of the nasal valve. The user slides a sealing nosepiece into one nostril until it forms a scal with the flexible soft tissue of the nostril opening, at which point, it mechanically expands the narrow slit-shaped part of the nasal triangular valve. The user then exhales through an attached mouthpiece. When exhaling into the mouthpiece against the resistance of the device, the soft palate (or velum) is automatically elevated by the positive oropharyngeal pressure, isolating the nasal cavity from the rest of the respiratory system. This mechanism enables release of liquid or powder particles into an air stream that enters one nostril, passes entirely around the nasal septum, and exits through the opposite nostril.

With sterile filling, the use of preservatives is not required in devices, but overfill is required resulting in a waste fraction similar to the metered-dose, multi-dose sprays. To cmit 100 μL, a volume of 125 μL is filled in the device (Pfeiffer/Aptar single-dose device) used for the intranasal migraine medications Imitrex® (sumatriptan) and Zomig® (zolmitriptan) and about half of that for a bi-dose design. Sterile drug products may be produced using aseptic processing or terminal sterilization. Terminal sterilization usually involves filling and scaling product containers under high-quality environmental conditions. Products are filled and scaled in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat or irradiation. In an aseptic process, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control.

### Methods of Treatment

Provided herein are methods of treatment of alcohol use disorder and related conditions comprising the intranasal administration of a therapeutically effective amount of naltrexone or a salt or hydrate thereof.

### Sinclair Method and Variations

The Sinclair Method is a treatment for AUD that employs pharmacological extinction—the use of an opioid antagonist, such as naltrexone, to turn the habit-forming behavior of drinking alcohol into a habit-erasing behavior. The effect returns a person's craving for alcohol to its pre-addiction state.

The method consists of taking an oral dose of naltrexone about 1, about 2, about 3, or about 4 hours before a subject ingests alcohol. This pre-ingestion dose of oral naltrexone disrupts the body's behavior and reward cycle thereby causing the person to want to drink less instead of more. Most significantly, studies have shown that this methodology is equally effective with or without therapy, so subjects can choose whether or not to combine this treatment method with other therapies without negatively impacting the actual physical results. Importantly, unlike the other currently approved medication treatments for AUD, the Sinclair Method calls for the use of oral naltrexone while the individual continues their normal drinking behavior. As a result, maintenance of the medication treatment protocol is expected to be much higher than abstinence alone.

Using the Sinclair Method, extinction of AUD can occur within 6 months. However, the efficacy of oral naltrexone is hampered by slow onset, very low bioavailability and high levels of the peripherally selective active metabolite 6-β-naltrexol, and the injectable form of naltrexone presents itself with the obvious difficulties associated with needles including, for example, the need for administration by a practitioner at regularly scheduled intervals. Thus, intranasal administration of naltrexone, and use of absorption enhancers, in a pre-primed, single or multi-use nasal spray pump should significantly improve results in the treatment of AUD. The timing of administration might also affect efficacy. An intranasal formulation of naltrexone absorbs quickly, providing fast onset of action and high bioavailability without the use of needles.

Accordingly, disclosed herein is a method of treatment of alcohol use disorder, or a related condition, in a subject, comprising administering to the subject an intranasal formulation comprising a therapeutically effective amount of naltrexone or a pharmaceutically acceptable salt thereof.

In certain embodiments, the intranasal formulation comprising naltrexone is administered prior to ingestion of alcohol.

In certain embodiments, the intranasal formulation comprising naltrexone is administered about 1 to about 2 hours prior to ingestion of alcohol. In certain embodiments, the intranasal formulation comprising naltrexone is administered about 1 hour prior to ingestion of alcohol. In certain embodiments, the intranasal formulation comprising naltrexone is administered about 0.5 to about 1 hours prior to ingestion of alcohol. In certain embodiments, the intranasal formulation comprising naltrexone is administered about 10 to about 30 minutes prior to ingestion of alcohol. In certain embodiments, the intranasal formulation comprising naltrexone is administered about 5 to about 10 minutes prior to ingestion of alcohol. In certain embodiments, the intranasal formulation comprising naltrexone is administered just before ingestion of alcohol.

In certain embodiments, the intranasal formulation comprising naltrexone is administered contemporaneously with the ingestion of alcohol.

In certain embodiments, the intranasal formulation comprising naltrexone is administered just after ingestion of alcohol. In certain embodiments, the intranasal formulation comprising naltrexone is administered within an hour after commencement of ingestion of alcohol.

It is expected that because intranasal naltrexone has a rapid uptake via the nasal mucosa and rapid appearance in the plasma, as evidenced by the studies below, intranasal administration will permit the subject to dose naltrexone much more immediately before, and even contemporaneously with or after, ingestion if alcohol, and experience benefits such as extinction, reduction in craving, etc. It is expected that absorption enhancers will further this effect.

In certain embodiments, the alcohol use disorder is alcohol abuse. In certain embodiments, the alcohol use disorder is alcohol dependence. In certain embodiments, the alcohol use disorder is alcoholism.

It is also expected that these methods will be effective in the treatment of other substance use disorders and reward-based disorders.

The methods disclosed herein may be achieved by administration of various embodiments of the formulations disclosed herein, for example above in the section “Pharmaceutical Formulations,” the embodiments above, and the Examples below. The formulations may be administered using devices known on the art, for example the devices disclosed herein in the section entitled “Nasal Drug Delivery Devices and Kits.”

Also provided herein are embodiments wherein any embodiment described above may be combined with any one or more other embodiment(s), provided the combination is not mutually exclusive.

### EXAMPLES

The following examples are included to demonstrate preferred embodiments of the invention. The following examples are presented only by way of illustration and to assist one of ordinary skill in using the invention. The examples are not intended in any way to otherwise limit the scope of the invention. Those of skill in the art should, in light of the present disclosure, appreciate that many changes can be made in the specific embodiments which are disclosed and still obtain a like or similar result without departing from the spirit and scope of the invention.

### Example 1: Intranasal Naltrexone Protocol for the Treatment of Alcohol Use Disorder

Individuals with alcohol use disorder (AUD) will be treated with intranasal naltrexone and examined for abstinence, reduced consumption of alcohol, and/or extinguished consumption of alcohol. Individuals with AUD are believed to release endogenous opioids upon the ingestion of alcohol. The binding of these opioids to receptors in the brain may be responsible for the positive reinforcing effects of alcohol. Drinking alcohol while the opioid antagonist naltrexone blocks the positive reinforcement from alcohol should extinguish alcohol drinking and craving.

In one example of a protocol, subjects (e.g., about 10-20) with AUD will make be admitted as in-patients to a study site. An initial visit serves the purpose of screening, to confirm the diagnosis and obtain informed consent. During their in-patient stay (e.g., one or more weeks), each subject will receive a placebo or intranasal dose of naltrexone followed by the consumption one or more alcoholic beverages. Naltrexone will be administered at the designated dose and by the designated method at about 0.25 to about 4 hrs before consumption of alcohol. One example of a dosing treatment is an intranasal formulation delivering about 1 to about 4 mg of naltrexone hydrochloride per administration, delivered by a single-or multi-use spray device. Another example of a dosing treatment is an intranasal formulation delivering a first dose of 3 mg of naltrexone hydrochloride in the morning, followed by subsequent doses of 3 mg of naltrexone hydrochloride throughout the day as needed by the patient. Yet another example of a dosing treatment is an intranasal formulation delivering up to 12 mg of naltrexone hydrochloride per day. The intranasal formulation of naltrexone may or may not contain an absorption enhancer, such as Intravail®.

It is expected that intranasal naltrexone will improve post-treatment suppression of alcohol intake. It is also expected that intranasal naltrexone will reduce alcohol cravings and the amount of time required for a subject to exhibit pharmacological extinction of alcohol cravings.

Approximately 1 hour prior to dosing, ECG, blood pressure, heart rate, and respiration rate will be measured and the time will be recorded. At approximately 1 and 4 hours after dosing, the ECG will be repeated and the time will be recorded. Vital signs including sitting (after 5 minutes) heart rate, blood pressure and respiration rate will be measured pre-dose and approximately 1 and 4 hours after each dose. Adverse events (AEs) will be recorded and treatment terminated if necessary. The nasal passage will be examined at pre-dose, 5 minutes, 30 minutes, 60 minutes, 4 hours, and 24 hours post-dose after intranasal administration only.

At screening, admission, and discharge, ECG, and vital signs will be checked once per day. Vital signs will also be checked once on the day after naltrexone administration. AEs will be assessed by spontaneous reports by subjects, by examination of the nasal mucosa, by measuring vital signs, ECG, and clinical laboratory parameters.

### Example 2: Pharmacokinetic Data Analysis

The non-compartmental pharmacokinetic (PK) parameters of naltrexone and 6β-naltrexol (Cₘₐₓ, Tₘₐₓ, AUCₒ₋ₜ, AUC_{o-∞}, t_{1/2}, λz, and apparent clearance (CL/F, naltrexone only) will be determined. PK parameters of various AUD treatment protocols (e.g., 4 mg intranasal with or without an absorption enhancer such as an alkylsaccharide; 50 mg oral tablet) will be compared with a 2 mg intramuscular (IM) dose of naltrexone. Dose-adjusted values for AUCs and Cₘₐₓ will be calculated. The relative extent of intranasal (IN) and oral absorption (PO) absorption will be estimated from the dose-corrected AUCs. Within an ANOVA framework, comparisons of IN-transformed PK parameters for IN and PO versus IM naltrexone treatments will be performed. The 90% confidence interval for the ratio (IN/IM and PO/IM) of the geometric least squares means of AUC and Cₘₐₓ parameters will be constructed for comparison of each treatment with IM naltrexone. These 90% confidence intervals will be obtained by exponentiation of the 90% confidence intervals for the difference between the least squares means based upon a log scale.

AEs will be coded using the most recent version of the Medical Dictionary for Regulatory Activities (MedDRA) preferred terms and will be grouped by system, organ, class (SOC) designation. The severity, frequency, and relationship of AEs to study drug will be presented by preferred term by SOC grouping. Separate summaries will be provided for the 4 study periods: after the administration of each dose of study drug up until the time of the next dose of study drug or clinic discharge. Listings of each individual AE including start date, stop date, severity, relationship, outcome, and duration will be provided.

Clinically significant changes in vital signs, ECG, and clinical laboratory parameters will be presented as counts and percentages by dosing session.

### Example 3: Dose Selection and Pharmaceutical Composition with Absorption Enhancers

Intranasal naltrexone may optionally be formulated with absorption-enhancing excipients.

One such excipient is the alkylsaccharide Intravail®. Concentrations of Intravail® in nasal formulations have generally been 0.1% and 0.2% by weight. The present study will use a concentration of 0.25% by weight of an alkylsaccharide. Concentrations of 25% Intravail® were non-irritating in the rabbit eye model. The oral “no observable effect level” was approximately 20,000 to 30,000 mg/kg body weight. While there is no comparable intranasal data, the essential lack of oral safety suggests that the amount of an alkylsaccharide needed for nasal toxicity would be much higher than the amount that will be administered in this study.

In the present study, a single dose of naltrexone was administered 4 ways: a) 4 mg IN in sterile water for injection; b) 4 mg IN in sterile water for injection with 0.25% Intravail®; c) 2 mg as an IM injection; and d) a 50-mg oral tablet. Intranasal administration is expected to increase the rate of absorption as compared to oral administration. Addition of Intravail® is expected to further increase the rate of absorption from the nasal passages.

### Example 4: Pharmacokinetic Evaluation of Intranasal Naltrexone

Sudy Goals. The purpose of this clinical study was twofold: to determine the pharmacokinetics of two intranasal formulations (4 mg with and without Intravail®) and one oral formulation (50 mg tablet) of naltrexone compared to a 2-mg intramuscular dose of naltrexone; and to determine the safety of intranasal naltrexone, particularly with respect to nasal irritation, such as inflammation (erythema, edema, and erosion) and bleeding. To that end, the study's primary endpoints were the pharmacokinetic parameters (Cₘₐₓ, Tₘₐₓ, AUC₀₋ₜ, and AUC_{0-inf}) of the IN and oral naltrexone formulations compared with an IM dose of 2 mg of naltrexone. Secondary endpoints included adverse events (AEs), vital signs (heart rate, sitting blood pressure, and respiration rate), electrocardiogram (ECG), clinical laboratory changes and nasal irritation using the nasal irritation scale.

Study design. Fourteen healthy volunteers were enrolled and completed all study drug administrations and blood collections for PK assessments. This was an in-patient open-label, crossover study involving approximately 14 healthy volunteers. Each subject received each naltrexone treatment: 4 mg IN (one 0.1 mL spray of a 40 mg/mL solution in one nostril), 4 mg plus Intravail® IN (one 0.1 mL spray of a 40 mg/mL solution containing 0.25% Intravail® in one nostril), 2 mg IM, and 50 mg oral tablet. Subjects stayed in the in-patient facility for 13 days to complete the entire study. Subjects were called 3 to 5 days after discharge to inquire concerning AEs and concomitant medications since discharge. Informed consent was obtained from all subjects, and all were screened for eligibility to participate in the study including medical history, physical examination, clinical chemistry, coagulation markers, hematology, infectious disease serology, urinalysis, urine drug and alcohol toxicology screen, vital signs and ECG.

On the day after clinic admission, subjects were administered study drug with a 3-day washout period between doses until all treatments had been administered. Blood was collected for analysis prior to dosing and approximately 2.5, 5, 10, 15, 20, 30, 45, 60 minutes and 2, 3, 4, 6, 8, 12, 16, 24, 30, 36, and 48 hours after study drug administration. On days of study drug administration, a 12-lead ECG was performed approximately 1 hour prior to dosing and at approximately 1 and 4 hours post-dose. Vital signs were measured pre-dose and approximately 1 and 4 hours post-dose.

On dosing days, the order of assessments were ECG, vital signs, then PK blood collection when scheduled at the same nominal times. The target time of the PK blood collection was considered the most critical and if the collection was more than ±1 minute from the scheduled time for the first 60 minutes of collections or more than ±5 minutes for the scheduled time points thereafter, this was considered a protocol deviation. ECG and vital signs were collected within the 10 minute period before the nominal time of blood collections. At screening, admission, and discharge, ECG, and vital signs were checked once per day. Vital signs were also checked once on the day after naltrexone administration. Clinical laboratory measurements were repeated after the last PK blood draw prior to clinic discharge. AEs were assessed by spontaneous reports by subjects, by examination of the nasal mucosa, by measuring vital signs, ECG, and clinical laboratory parameters.

Inclusion and exclusion criteria: 1. Males and females 18 to 55 years of age, inclusive were included in this study. Written informed consent was required. Subject had to:
have body mass index (BMI) ranging from 18 to 30 kg/m², inclusive;
have adequate venous access;
have no clinically significant concurrent medical conditions determined by medical history, physical examination, clinical laboratory examination, vital signs, and 12-lead ECG;
agree to use an acceptable method of contraception, other than oral contraceptives, throughout the study and for 90 days after the last study drug administration (30 days for women); and
agree not to ingest alcohol, drinks containing xanthine >500 mg/day (e.g., Coca-Cola®, tea, coffee, etc.), or grapefruit/grapefruit juice or participate in strenuous exercise 72 hours prior to admission through the last blood draw of the study.

Exclusion criteria included:
any IN conditions including abnormal nasal anatomy, nasal symptoms (i.e., blocked and/or runny nose, nasal polyps, etc.);
having a product sprayed into the nasal cavity prior to screening and drug administration;
having been administered an investigational drug within 30 days prior to Day-1;
having taken prescribed or over-the-counter medications, dietary supplements, herbal products, vitamins, or recent use of opioid analgesics for pain relief (within 14 days of last use of any of these products);
a positive urine drug test for alcohol, opioids, cocaine, amphetamine, methamphetamine, benzodiazepines, tetrahydrocannabinol (THC), barbiturates, or methadone at screening or admission;
previous or current opioid, alcohol, or other drug dependence (excluding nicotine and caffeine), based on medical history;
consumption of greater than 20 cigarettes per day on average, in the month prior to screening, or would be unable to abstain from smoking (or use of any nicotine-containing substance) for at least one hour prior to and 2 hours after naltrexone dosing;
systolic blood pressure less than 90 mm Hg or greater than 140 mm Hg; diastolic blood pressure less than 55 mmHg or greater than 90 mmHg; respiratory rate less than 8 respirations per minute or greater than 20 respirations per minute;
on standard 12-lead ECG, a QTcF interval >440 msec for males and >450 msec for females; significant acute or chronic medical disease (investigator judgment);
a likely need for concomitant medication treatment during the study;
donated or received blood or underwent plasma or platelet apheresis within the 60 days prior to Day-1;
female who is pregnant, breast feeding, or plans to become pregnant during the study period or within 30 days after the last naltrexone administration;
positive test for hepatitis B surface antigen (HBsAg), hepatitis C virus antibody (HCVAb) or human immunodeficiency virus antibody (HIVAb) at screening;
current or recent (within 7 days prior to screening) upper respiratory tract infection; and
abnormal liver function test (ALT, AST, total bilirubin) >1.5 times upper limit of normal

Study Drugs and Dosing. Naltrexone hydrochloride (HCl) was obtained from Mallinckrodt Pharmaceuticals. The IN (40 mg/mL) formulations were made by the staff pharmacist at Vince & Associates; the vehicle for the IN formulations was sterile water for injection. The IM formulation (2 mg/mL) was made by the staff pharmacist at Vince & Associates; the vehicle was sterile saline for injection. IN naltrexone was administered using an Aptar multi-dose device with the subject in a reclined position (approximately 45 degrees). The subject was instructed not to breathe through the nose when the IN dose of naltrexone was administered. Naltrexone HCl for the IM injection was administered with a 23-g needle as a single 1-mL injection into the gluteus maximus muscle. Naltrexone HCl for oral administration (50 mg tablet) was sourced from a commercial supplier and administered with 240 mL water.

Naltrexone was administered on Days 1, 4, 7, and 10, in the following order: 4 mg naltrexone IN, 4 mg naltrexone plus Intravail® IN, 2 mg IM, and 50 mg oral. Subjects stayed in the in-patient facility for 13 days to complete the entire study and were discharged 2 days after the fourth dose.

PK Assessments. Blood (4 mL) was collected in sodium heparin containing tubes for PK analysis prior to dosing and 2.5, 5, 10, 1.5, 20, 30, 45, 60 minutes and 2, 3, 4, 6, 8, 12, 16, 24, 30, 36, and 48 hours after the start of study drug administration. Plasma was separated from whole blood and stored frozen at ≤20° C. until assayed. Naltrexone and 6β-naltrexol plasma concentrations were determined by liquid chromatography with tandem mass spectrometry at XenoBiotic laboratories, Inc., Plainsboro, New Jersey.

Safety Assessments. Heart rate, blood pressure, and respiration rate were recorded approximately 1 hour before naltrexone dosing and approximately 1 and 4 hours after dosing. A 12-lead ECG was obtained approximately 1 hour before and 1 and 4 hours after each naltrexone dose. ECG and vital signs was performed within the 10 minute period before the nominal time for post-dose blood collections. AEs were recorded from the start of study drug administration until clinic discharge. AEs were recorded relative to each dosing session to attempt to establish a relationship between the AE and type of naltrexone dose administered. An examination of the nasal passage was conducted at Day-1 to establish eligibility and at pre-dose, 5 minutes, 30 minutes, 60 minutes, 4 hours, and 24 hours post IN naltrexone administration to evaluate evidence of irritation to the nasal mucosa after IN administration only.

Analysis. Non-compartmental PK parameters of naltrexone and 6β-naltrexol, including, Tₘₐₓ, AUC₀₋ₜ, and AUC_{0-inf}, t_{1/2}, λz, and apparent clearance (CL/F, naltrexone only), was determined. Pharmacokinetic parameters (Cₘₐₓ, Tₘₐₓ and AUCs) for IN and PO naltrexone were compared with those for IM naltrexone. Dose-adjusted values for AUCs and Cₘₐₓ were calculated. The relative extent of IN and PO absorption (IN and PO versus IM) will be estimated from the dose-corrected AUCs. Within an ANOVA framework, comparisons of ln-transformed PK parameters (Cₘₐₓ and AUC) for IN and PO versus IM naltrexone treatments were performed. The 90% confidence interval for the ratio (IN/IM and PO/IM) of the geometric least squares means of AUC and Cₘₐₓ parameters were constructed for comparison of each treatment with IM naltrexone. These 90% CIs were obtained by exponentiation of the 90% CIs for the difference between the least squares means based upon an In scale.

Results. Results are shown below in Tables 1-5.

TABLE 1
Mean (SD) concentrations of naltrexone following a single IN, IM or oral
administration to healthy subjects.

| | Treatment | | | | | | | |
|---|---|---|---|---|---|---|---|---|
| Hour | 4 mg IN | | 4 mg IN + 0.25% Intravail ^{®} | | 2 mg IN | | 50 mg Oral | |
| 0 | 0 | 0 | 0 | 0 | 0 | 0 | 0 | 0 |
| 0.042 | 0.117 | (0.17) | 1.15 | (0.919) | 0.678 | (1.69) | 0 | 0 |
| 0.083 | 1.51 | (1.62) | 11.9 | (9.69) | 1.04 | (1.26) | 0.109 | (0.232) |
| 0.17 | 3.4 | (3.86) | 12.1 | (6.36) | 2.97 | (2) | 0.851 | (1.5) |
| 0.25 | 4.36 | (3.71) | 10.4 | (3.93) | 3.45 | (1.58) | 2.5 | (3.54) |
| 0.33 | 4.46 | (3.62) | 9.81 | (2.41) | 3.58 | (1.46) | 4.75 | (4.71) |
| 0.5 | 4.08 | (1.99) | 7.19 | (2.08) | 3.43 | (1.06) | 7.16 | (4.69) |
| 0.75 | 3.39 | (1.46) | 5.46 | (1.48) | 3.02 | (0.749) | 6.9 | (3.54) |
| 1 | 3.19 | (1.51) | 4.55 | (1.78) | 2.73 | (0.676) | 6.34 | (2.78) |
| 2 | 2.33 | (0.832) | 3.07 | (1.27) | 2.35 | (0.698) | 5.22 | (1.73) |
| 3 | 1.5 | (0.633) | 2 | (0.885) | 1.79 | (0.491) | 3.66 | (1.76) |
| 4 | 1.02 | (0.369) | 1.25 | (0.465) | 1.3 | (0.341) | 2.39 | (1.16) |
| 6 | 0.418 | (0.193) | 0.536 | (0.188) | 0.584 | (0.185) | 1.13 | (0.462) |
| 8 | 0.22 | (0.0941) | 0.267 | (0.105) | 0.242 | (0.0803) | 0.596 | (0.426) |
| 12 | 0.0641 | (0.021) | 0.0726 | (0.028) | 0.0626 | (0.0269) | 0.3 | (0.183) |
| 16 | 0.0214 | (0.0131) | 0.0226 | (0.0165) | 0.0101 | (0.0132) | 0.141 | (0.104) |
| 24 | 0.00462 | (0.0117) | 0 | 0 | 0 | 0 | 0.0657 | (0.0528) |
| 30 | 0.00187 | (0.00674) | 0 | 0 | 0 | 0 | 0.0345 | (0.0224) |
| 36 | 0 | 0 | 0 | 0 | 0 | 0 | 0.0207 | (0.0255) |
| 48 | 0.0018 | (0.0065) | 0 | 0 | 0 | 0 | 0 | 0 |

TABLE 2
Mean CV % PK Parameters for Naltrexone Following Administration to Healthy Subjects

| | | 4 mg IN | | |
|---|---|---|---|---|
| | | plus 0.25% | | |
| PK Parameter | 4 mg IN^{a} | Intravail ® ^{b} | 2 mg IM^{c} | 50 mg Oral^{c} |
| Cₘₐₓ (ng/mL) | 5.35 (66.8) | 15.7 (52.0) | 4.10 (34.0) | 9.34 (31.8) |
| Cₘₐₓ/Dose (ng/mL/mg) | 1.48 (66.8) | 4.35 (52.0) | 2.27 (34.0) | 0.206 (31.8) |
| Tₘₐₓ (h) ^{d} | 0.50 (0.17, | 0.17 (0.083, | 0.33 (0.17, | 0.50 (0.33, |
| | 2.00) | 0.33) | 1.00) | 3.00) |
| AUC₀₋ₜ (h · ng/mL) | 11.9 (34.1) | 18.3 (31.2) | 12.1 (25.5) | 26.5 (32.3) |
| AUC₀₋ₜ/Dose (h · ng/mL/mg) | 3.28 (34.1) | 5.07 (31.2) | 6.71 (25.5) | 0.587 (32.3) |
| AUC_{0-inf} (h · ng/mL) | 12.0 (33.7) | 18.5 (31.0) | 12.3 (25.6) | 26.9 (31.8) |
| AUC_{0-inf}/Dose (h · ng/mL/mg) | 3.32 (33.7) | 5.10 (31.0) | 6.78 (25.6) | 0.594 (31.8) |
| AUCₑₓₜᵣₐₚ (%) | 1.09 (57.0) | 0.707 (44.0) | 1.01 (71.7) | 1.38 (70.1) |
| CL/F (L/h) | 330 (28.9) | 214 (33.6) | 154 (19.0) | 1890 (41.4) |
| λ_{z} (1/h) | 0.281 (15.1) | 0.317 (15.1) | 0.361 (16.8) | 0.122 (38.0) |
| t_{1/2} (h) | 2.52 (14.9) | 2.23 (14.9) | 1.97 (15.5) | 6.41 (36.6) |
| Fᵣₑₗ | 0.481 (36.1)^{c} | 0.783 (17.7)^{c} | NA | 0.0903 (37.0) |

NA = Not applicable;
Frel = Bioavailability relative to IM dose, calculated as ratio of AUCinf/Dose for IN or PO route relative to IM route.
^{a}N = 13;
^{b} N = 12;
^{c}N = 10;
^{d} Median (minimum, maximum)

Following IN administration of 4 mg naltrexone, the mean concentration at 2.5 minutes postdose was 0.117 ng/mL. When 0.25% Intravail® was added to the formulation, the mean concentration was 10 times greater (1.15 ng/ml) at 2.5 minutes. At 5 minutes postdose, the mean concentrations of naltrexone with and without Intravail® were 11.9 ng/mL and 1.51 ng/mL, respectively, an 8-fold difference. The addition of 0.25% Intravail® to the IN formulation decreased median Tₘₐₓ from 30 minutes to 10 minutes and increased Cₘₐₓ almost 3-fold (15.7 versus 5.35 ng/mL). Overall exposure as measured by AUCo-inf increased by 54%, indicating that the main effect of Intravail® was to increase the rate of absorption more than the extent.

The mean plasma concentrations of naltrexone at 2.5 and 5 minutes after administration of 2 mg naltrexone IM were 0.678 ng/ml and 1.04 ng/mL, respectively. The mean Cₘₐₓ value of 4.10 ng/ml 20 minutes after the 2 mg IM dose was 23% less than after the 4 mg IN dose and 74% less compared to when Intravail® was part of the IN formulation.

The mean Cₘₐₓ value after the oral dose was 9.34 ng/mL, which was less than observed after the IN dose with Intravail® even though 50 mg was administered orally compared to only 4 mg IN.

The mean terminal phase half-life (t½) of naltrexone was 1.97 hours to 2.52 hours after IM and IN administration. The t½ was 6.41 hours after the oral dose.

When AUC_{0-inf} values were corrected for dose, the relative bioavailability of naltrexone after the IN doses with and without 0.25% Intravail® was 78% and 48%, respectively, compared to the IM administration. The relative bioavailability for the oral dose was only 9%, indicating extensive first pass metabolism by the gastrointestinal tract and liver.

Statistical analysis of dose-adjusted PK parameters suggested exposure for the IN dose was approximately 48% or 60% of the IM dose on a per mg basis, in terms of geometric least-squares mean (GM) dose-adjusted AUC and Cₘₐₓ, respectively. IN administration of naltrexone with 0.25% Intravail® resulted in dose-adjusted exposure that was higher than the IM route in terms of Cₘₐₓ (geometric least-squares mean ratio between treatments [GMR] of 188%) and lower in terms of AUC (GMR of 76%). For the oral route, the GMR for dose-adjusted naltrexone exposure was approximately 9% of the IM dose.

TABLE 3
Mixed-Effects ANOVA Results for Naltrexone Pharmacokinetic Parameters
Following Intranasal or Oral Administration vs. Intramuscular Administration to Healthy Subjects

| | | | 90% CI of | |
|---|---|---|---|---|
| | | GMR | GMR (%) | |
| PK Parameter | Comparison (2 mg IM Reference) | (%) | Lower | Upper |
| Cmax (ng/mL) | 4 mg IN vs 2 mg IM | 121 | 91.1 | 160 |
| | 4 mg IN plus Intravail® | 377 | 321 | 442 |
| | vs 2 mg IM | | | |
| | 50 mg PO vs 2 mg IM | 231 | 190 | 282 |
| AUC₀₋ₜ | 4 mg IN vs 2 mg IM | 96.4 | 82.6 | 112 |
| (h · ng/mL) | 4 mg IN plus Intravail® | 152 | 136 | 169 |
| | vs 2 mg IM | | | |
| | 50 mg PO vs 2 mg IM | 221 | 182 | 268 |
| AUC_{0-inf} | 4 mg IN vs 2 mg IM | 96.4 | 82.7 | 112 |
| (h · ng/mL) | 4 mg IN plus Intravail® | 151 | 136 | 168 |
| | vs 2 mg IM | | | |
| | 50 mg PO vs 2 mg IM | 221 | 183 | 268 |
| Cmax/Dose | 4 mg IN vs 2 mg IM | 60.4 | 45.5 | 80.2 |
| (ng/mL/mg) | 4 mg IN plus Intravail® | 188 | 161 | 221 |
| | vs 2 mg IM | | | |
| | 50 mg PO vs 2 mg IM | 9.3 | 7.6 | 11.3 |
| AUC₀₋ₜ /Dose | 4 mg IN vs 2 mg IM | 48.2 | 41.3 | 56.2 |
| (h · ng/mL/mg) | 4 mg IN plus Intravail® | 75.9 | 68.1 | 84.6 |
| | vs 2 mg IM | | | |
| | 50 mg PO vs 2 mg IM | 8.8 | 7.3 | 10.7 |
| AUC_{0-inf}Dose | 4 mg IN vs 2 mg IM | 48.2 | 41.4 | 56.2 |
| (h · ng/mL/mg) | 4 mg IN plus Intravail® | 75.7 | 68 | 84.2 |
| | vs 2 mg IM | | | |
| | 50 mg PO vs 2 mg IM | 8.9 | 7.3 | 10.7 |

GMR = Geometric least-squares mean ratio between treatments (expressed as percentage of reference)

The mean Cₘₐₓ values of 6β-naltrexol were 1.5 ng/mL after the IM administration and approximately 3 ng/ml after the IN administration; Cmax was 90.7 ng/ml after the 50 mg oral dose (Table 2-3). When adjusted for the administered dose, the Cmax values were similar for the IN and IM doses (0.833 and 0.838 ng/ml/mg) but approximately 2-fold higher (2.00 ng/mL/mg) after oral administration.

Values of AUC_{0-inf} also were increased considerably after the oral dose in comparison to the IN and IM doses (675 h·ng/ml and 44.0 to 27.1 h·ng/mL, respectively). The greater extent of first pass metabolism of naltrexone was evident in the ratio of AUC0-inf for 6β-naltrexol compared to that of naltrexone: after the IN and IM doses, the ratio was approximately 2.2 to 3.7 while it was 25 after the oral dose.

The mean to, of the metabolite was 12.4 to 13.9 hours and was independent of the route of administration.

TABLE 4
Mean (SD) concentrations of 6β-naltrexol following a single IN, IM or oral administration to healthy subjects.

| | Treatment | | | | | | | |
|---|---|---|---|---|---|---|---|---|
| Hour | 4 mg IN | | 4 mg IN + 0.25% Intravail ^{®} | | 2 mg IN | | 50 mg Oral | |
| 0 | 0 | 0 | 0.0682 | (0.0257) | 0.0661 | (0.0256) | 0.0454 | (0.0141) |
| 0.042 | 0 | 0 | 0.082 | (0.0378) | 0.0627 | (0.0248) | 0.0448 | (0.0202) |
| 0.083 | 0.0321 | (0.0432) | 0.238 | (0.146) | 0.12 | (0.117) | 1.4 | (3.49) |
| 0.17 | 0.196 | (0.196) | 0.994 | (0.558) | 0.283 | (0.281) | 14.2 | (31) |
| 0.25 | 0.45 | (0.448) | 1.86 | (0.763) | 0.454 | (0.293) | 31.6 | (47.3) |
| 0.33 | 0.693 | (0.624) | 2.55 | (0.918) | 0.677 | (0.385) | 45.5 | (43.7) |
| 0.5 | 1.11 | (0.559) | 2.84 | (0.748) | 0.852 | (0.328) | 68.7 | (41.3) |
| 0.75 | 1.82 | (1.16) | 2.93 | (0.757) | 1.08 | (0.452) | 60.8 | (28.2) |
| 1 | 1.83 | (0.815) | 2.73 | (0.481) | 1.1 | (0.404) | 58.6 | (18.2) |
| 2 | 2.68 | (0.842) | 2.9 | (0.767) | 1.39 | (0.462) | 54 | (16.3) |
| 3 | 2.61 | (0.793) | 2.61 | (0.708) | 1.48 | (0.402) | 45.8 | (15.2) |
| 4 | 2.37 | (0.669) | 2.45 | (0.598) | 1.46 | (0.388) | 38 | (12.2) |
| 6 | 1.97 | (0.554) | 2.03 | (0.399) | 1.3 | (0.252) | 28.5 | (7.52) |
| 8 | 1.62 | (0.418) | 1.67 | (0.27) | 1.08 | (0.165) | 22.2 | (5.51) |
| 12 | 1.26 | (0.299) | 1.25 | (0.176) | 0.81 | (0.131) | 15.4 | (2.85) |
| 16 | 0.919 | (0.229) | 0.923 | (0.155) | 0.595 | (0.115) | 11.4 | (1.78) |
| 24 | 0.602 | (0.194) | 0.618 | (0.145) | 0.365 | (0.0808) | 8.14 | (2.09) |
| 30 | 0.418 | (0.114) | 0.457 | (0.116) | 0.255 | (0.0666) | 5.93 | (1.85) |
| 36 | 0.292 | (0.0868) | 0.312 | (0.0862) | 0.18 | (0.0478) | 4.35 | (1.54) |
| 48 | 0.184 | (0.0645) | 0.175 | (0.0623) | 0.106 | (0.0329) | 2.43 | (0.882) |

TABLE 5
Mean (CV %) PK Parameters for 6β-Naltrexol Following Administration to Healthy Subjects

| | | 4 mg IN | | |
|---|---|---|---|---|
| | | plus 0.25% | | |
| PK Parameter | 4 mg IN^{a} | Intravail ® ^{b} | 2 mg IM^{c} | 50 mg Oral^{c} |
| Cₘₐₓ (ng/mL) | 3.01 (33.2) | 3.29 (23.7) | 1.52 (26.8) | 90.7 (30.3) |
| Cₘₐₓ/Dose (ng/mL/mg) | 0.833 (33.2) | 0.908 (23.7) | 0.838 (26.8) | 2.00 (30.3) |
| Tₘₐₓ (h)^{d} | 2.00 (0.75, | 0.75 (0.25, | 3.00 (0.75, | 0.63 (0.25, |
| | 6.0) | 4.00) | 4.00) | 3.00) |
| AUC₀₋ₜ (h · ng/mL) | 40.3 (23.3) | 43.0 (17.4) | 25.1 (18.3) | 614 (19.5) |
| AUC₀₋ₜ/Dose | 11.1 (23.3) | 11.9 (17.4) | 13.9 (18.3) | 13.6 (19.5) |
| (h · ng/mL/mg) | | | | |
| AUC_{0-inf} (h · ng/mL) | 44.0 (23.1) | 46.3 (18.3) | 27.1 (19.0) | 675 (19.9) |
| AUC_{0-inf}/Dose | 12.2 (23.1) | 12.8 (18.3) | 15.0 (19.0) | 14.9 (19.9) |
| (h · ng/mL/mg) | | | | |
| AUCₑₓₜᵣₐₚ (%) | 8.57 (46.1) | 7.02 (37.3) | 7.02 (29.4) | 8.79 (57.3) |
| λ_{z} (1/h) | 0.0530 (21.8) | 0.0553 (15.1) | 0.0570 (14.8) | 0.0510 (15.8) |
| t½ (h) | 13.7 (22.7) | 12.8 (14.6) | 12.4 (13.2) | 13.9 (15.9) |

*Median (Min, Max) statistics presented for Tmax. All other values presented as: Mean (Percent coefficient of variation);
^{a}: N = 13;
^{b}: N = 12;
^{c}: N = 10;
^{d}: Median (minimum, maximum)

With the exception of the mean Cₘₐₓ of naltrexone following the 4 mg IN dose, which was approximately 2-fold higher in females compared to males, there was no clinically meaningful difference between the sexes for the PK parameters of either naltrexone or 6β-naltrexol following IN, IM, or PO administration.

Safety. In total, 10 of 14 subjects (71%) in the safety population experienced at least one AE (any dosing period, any relationship to drug). The most frequent AEs were of the Nervous System Disorders SOC (7 subjects, 50%), and dizziness was the most frequent AE regardless of severity or attribution (5 subjects, 36%). No severe AEs were observed, and only one moderate AE was observed, a case of dizziness after the first dose (4 mg IN) that was considered related to the study agent. Three subjects experienced AEs that were unexpected (UAE, defined as AEs that were not described with respect to nature, severity, or frequency in the current product package insert): two UAEs were considered unrelated to the study agent and one treatment-related UAE of mild syncope after administration of the Day 1 dose (4 mg IN). Three subjects were discontinued from the study due to AEs (hypertension, syncope, and out-of-range pre-dose vital signs).

### Example 5: Formulations of Intranasal Naltrexone

The following tables set forth examples of formulations of naltrexone for intranasal administration for the treatment of disorders. Table 6 sets forth simple aqueous solution formulations such as those used in the experiment above, to be dispensed in increments of about 100 μL.

TABLE 6

| | Naltrexone | | μL | |
|---|---|---|---|---|
| | HCl, | Absorption | per | Conc., |
| Ex. | dose (mg) | Enhancer | dose | mg/mL |
| 1 | 1.2 | Intravail 0.25% | 50 | 24 |
| 2 | 1.2 | Intravail 0.25% | 100 | 12 |
| 3 | 1.2 | Intravail 0.25% | 150 | 8 |
| 4 | 1.6 | Intravail 0.25% | 50 | 32 |
| 5 | 1.6 | Intravail 0.25% | 100 | 16 |
| 6 | 1.6 | Intravail 0.25% | 150 | 10.7 |
| 7 | 2 | Intravail 0.25% | 50 | 40 |
| 8 | 2 | Intravail 0.25% | 100 | 20 |
| 9 | 2 | Intravail 0.25% | 150 | 13.3 |
| 10 | 3 | Intravail 0.25% | 100 | 30 |
| 11 | 3 | Intravail 0.25% | 150 | 20 |
| 12 | 4 | Intravail 0.25% | 100 | 40 |
| 13 | 4 | Intravail 0.25% | 150 | 26.7 |

Table 7 sets forth formulations for intranasal administration in 100 μL of an aqueous solution including excipients such as an isotonicity agent, a stabilizing agent, and/or a compound which acts as a preservative or surfactant. EDTA stands for disodium edetate and BZK stands for benzalkonium chloride.

TABLE 7

| | Naltrexone | Absorption | Isotonicity | Stabilizing | Preservative/ |
|---|---|---|---|---|---|
| Ex. | HCl | Enhancer | Agent | Agent | Surfactant |
| 14 | 1.2 mg | Intravail 0.25% | NaCl 0.74% | EDTA 0.3% | BZK 0.02% |
| 15 | 1.2 mg | Intravail 0.25% | NaCl 0.74% | EDTA 0.3% | BZK 0.01% |
| 16 | 1.2 mg | Intravail 0.25% | NaCl 0.74% | EDTA 0.2% | BZK 0.02% |
| 17 | 1.2 mg | Intravail 0.25% | NaCl 0.74% | EDTA 0.2% | BZK 0.01% |
| 18 | 1.6 mg | Intravail 0.25% | NaCl 0.74% | EDTA 0.3% | BZK 0.02% |
| 19 | 1.6 mg | Intravail 0.25% | NaCl 0.74% | EDTA 0.3% | BZK 0.01% |
| 20 | 1.6 mg | Intravail 0.25% | NaCl 0.74% | EDTA 0.2% | BZK 0.02% |
| 21 | 1.6 mg | Intravail 0.25% | NaCl 0.74% | EDTA 0.2% | BZK 0.01% |
| 22 | 2 mg | Intravail 0.25% | NaCl 0.74% | EDTA 0.3% | BZK 0.02% |
| 23 | 2 mg | Intravail 0.25% | NaCl 0.74% | EDTA 0.3% | BZK 0.01% |
| 24 | 2 mg | Intravail 0.25% | NaCl 0.74% | EDTA 0.2% | BZK 0.02% |
| 25 | 2 mg | Intravail 0.25% | NaCl 0.74% | EDTA 0.2% | BZK 0.01% |
| 26 | 3 mg | Intravail 0.25% | NaCl 0.74% | EDTA 0.3% | BZK 0.02% |
| 27 | 3 mg | Intravail 0.25% | NaCl 0.74% | EDTA 0.3% | BZK 0.01% |
| 28 | 3 mg | Intravail 0.25% | NaCl 0.74% | EDTA 0.2% | BZK 0.02% |
| 29 | 3 mg | Intravail 0.25% | NaCl 0.74% | EDTA 0.2% | BZK 0.01% |
| 30 | 1.2 mg | Intravail 0.18% | NaCl 0.74% | EDTA 0.3% | BZK 0.02% |
| 31 | 1.2 mg | Intravail 0.18% | NaCl 0.74% | EDTA 0.3% | BZK 0.01% |
| 32 | 1.2 mg | Intravail 0.18% | NaCl 0.74% | EDTA 0.2% | BZK 0.02% |
| 33 | 1.2 mg | Intravail 0.18% | NaCl 0.74% | EDTA 0.2% | BZK 0.01% |
| 34 | 1.6 mg | Intravail 0.18% | NaCl 0.74% | EDTA 0.3% | BZK 0.02% |
| 35 | 1.6 mg | Intravail 0.18% | NaCl 0.74% | EDTA 0.3% | BZK 0.01% |
| 36 | 1.6 mg | Intravail 0.18% | NaCl 0.74% | EDTA 0.2% | BZK 0.02% |
| 37 | 1.6 mg | Intravail 0.18% | NaCl 0.74% | EDTA 0.2% | BZK 0.01% |
| 38 | 2 mg | Intravail 0.18% | NaCl 0.74% | EDTA 0.3% | BZK 0.02% |
| 39 | 2 mg | Intravail 0.18% | NaCl 0.74% | EDTA 0.3% | BZK 0.01% |
| 40 | 2 mg | Intravail 0.18% | NaCl 0.74% | EDTA 0.2% | BZK 0.02% |
| 41 | 2 mg | Intravail 0.18% | NaCl 0.74% | EDTA 0.2% | BZK 0.01% |
| 42 | 3 mg | Intravail 0.18% | NaCl 0.74% | EDTA 0.3% | BZK 0.02% |
| 43 | 3 mg | Intravail 0.18% | NaCl 0.74% | EDTA 0.3% | BZK 0.01% |
| 44 | 3 mg | Intravail 0.18% | NaCl 0.74% | EDTA 0.2% | BZK 0.02% |
| 45 | 3 mg | Intravail 0.18% | NaCl 0.74% | EDTA 0.2% | BZK 0.01% |

Also provided are examples 1A-45A which additionally contain an amount of hydrochloric acid sufficient to achieve a pH of 3.5-5.5. The acid should be pharmaceutically acceptable, for example, hydrochloric acid.

### Example 6: Intranasal Naltrexone Formulation Experiments

A series of experiments were performed to identify formulations of naltrexone which would make pharmaceutically appropriate intranasal products as disclosed herein. The formulations were designed to have at least some, and in certain embodiments, all, of the following properties:
containing an efficacious amount of naltrexone;
naltrexone quickly absorbed after administration, yielding a good Cₘₐₓ and relatively short Tₘₐₓ;
would form a solution at room temperature, and remain a solution at cold storage conditions, or revert to solution when removed from cold storage;
would not be contaminated with microorganisms;
would be storage-stable, would not form or acquire undesirable impurities, and/or would not discolor over time; and n
on-irritating to nasal membrane tissue.

A range of formulations were tested with the following observations. A 50 mg/mL preparation of naltrexone HCl was cloudy at room temperature, indicating incomplete dissolution; the solubility limit of naltrexone hydrochloride in solution at ambient temperature was determined to be approximately 40 mg/mL. Several preparations were made at 30 mg/mL thereafter, which additionally contained fixed amounts of (dodecyl maltoside 0.25%) and NaCl (about 0.74%), and varying amounts of preservative (benzalkonium chloride, 0-0.02%) and stabilizing agent (EDTA, 0-0.3%). Crystallization behavior and discoloration were visually monitored. All of these formulations developed crystals when stored under refrigerated conditions. Naltrexone solutions tend to turn yellow with time, over the course of 0 to 3 months; formulations, containing 0.2% or 0.3% EDTA, did not form yellow solutions, whereas a formulation containing 0% or 0.1% EDTA did begin to yellow. The 0.3% EDTA formulation appeared to resist yellowing for the longest period of time. Thereafter, a range of co-solvents were assessed for their potential to reduce crystallization of drug out of solution. Polyethylene glycol (PEG), propylene glycol, and benzyl alcohol were tested. PEG-containing formulations did not prevent crystallization; propylene glycol containing formulations prevented crystallization but resulted in high osmolality, which would be expected to result in irritation of nasal membrane tissue; benzyl alcohol containing formulations prevented crystallization at low concentration. Total solid content was observed to be related to crystallization, and reducing the amount of naltrexone hydrochloride to produce the lower concentrations and the amount of NaCl in order to adjust for osmolality assisted in keeping the formulation in solution when stored under refrigerated conditions. Four formulations of naltrexone suitable for intranasal administration are given below in Example 7.

### Example 7: Additional Formulations of Intranasal Naltrexone

The following are additional examples of batch formulations of naltrexone, which each make 2000 liquid grams of the give formulation for intranasal administration for the treatment of disorders, including Alcohol Use Disorder, which may be dispensed in increments of, e.g., about 100 μL.

Examples below were prepared as follows. To a tared 4 L batch container with a stir bar, 1800.0 grams of water for injection (WFI) was added and mixing initiated. Either 24.0 g, 32.0 g, 40.0 g or 60.0 g of naltrexone hydrochloride (NH) was added, the NH container rinsed with four (4) 5 mL WFI rinses, and mixed until the NH was visually dissolved. While mixing, 5.0 g DDM was added and mixed until visually dissolved; then the same procedure with 6.0 g EDTA. Next, an initial quantity of 12.6 g, 11.7 g, 10.5 g, or 7.9 g of NaCl was added depending on the formulation concentration; NaCl was added intermittently and mixed between additions until visually dissolved. Next, while mixing, 40.0 g of BZK 1% stock solution was added, the BZK 1% stock solution container rinsed with a 10 mL of rinse, and mixed until the BZK was visually dissolved. Finally, pH of the solution was measured using a calibrated pH meter, and if above 5.5, adjusted accordingly utilizing 10% HCl until it was between 3.5 and 5.5, with a target pH of 5.0. This was done by dropwise addition and mixing of 3-5 min between drops, then water added Q.S. to approximately 2000.0 g and stirred for 5 min.

Formulation 49: 12 mg/mL (0.3% EDTA & 0.02% BZK)

| Ingredient | w/w % | Formula Quantity (g) |
|---|---|---|
| Water for Injection | 95.62 | 1912.4 |
| Naltrexone Hydrochloride | 1.2 | 24 |
| Sodium Chloride | 0.63 | 12.6 |
| Disodium EDTA Dihydrate | 0.3 | 6 |
| Benzalkonium Chloride (1%) | 2 | 40 |
| DDM (Dodecyl Maltoside) | 0.25 | 5 |

Formulation 50: 16 mg/mL (0.3% EDTA & 0.02% BZK)

| Ingredient | w/w | Formula Quantity (g) |
|---|---|---|
| Water for Injection | 95.265 | 1905.3 |
| Naltrexone Hydrochloride | 1.6 | 32 |
| Sodium Chloride | 0.585 | 11.7 |
| Disodium EDTA Dihydrate | 0.3 | 6 |
| Benzalkonium Chloride (1%) | 2 | 40 |
| DDM (Dodecyl Maltoside) | 0.25 | 5 |

Formulation 51: 20 mg/mL (0.3% EDTA & 0.02% BZK)

| Ingredient | w/w | Formula Quantity (g) |
|---|---|---|
| Water for Injection | 94.912 | 1898.24 |
| Naltrexone Hydrochloride | 2 | 40 |
| Sodium Chloride | 0.538 | 10.76 |
| Disodium EDTA Dihydrate | 0.3 | 6 |
| Benzalkonium Chloride (1%) | 2 | 40 |
| DDM (Dodecyl Maltoside) | 0.25 | 5 |

Formulation 52: 30 mg/mL (0.3% EDTA & 0.02% BZK)

| Ingredient | w/w | Formula Quantity (g) |
|---|---|---|
| Water for Injection | 94.055 | 1881.1 |
| Naltrexone Hydrochloride | 3 | 60 |
| Sodium Chloride | 0.395 | 7.9 |
| Disodium EDTA Dihydrate | 0.3 | 6 |
| Benzalkonium Chloride (1%) | 2 | 40 |
| DDM (Dodecyl Maltoside) | 0.25 | 5 |

The formulations above may be tested according to the procedures above in Examples A-E and according to methods known in the art; certain of these formulations have been tested in Example 8 below. It is expected that the pharmacokinetic properties of these formulations will be consistent with those of an effective medication to treat opioid overdose as well as to use in ‘as needed’ fashion to treat multiple substance use disorders, exemplified by alcohol use disorder, and other reward-based disorders. These properties include a rapid onset (short Tₘₐₓ), high plasma concentration, and short half-life relative to oral administration that can be achieved with these formulations.

### Example 8: Pharmacokinetic Evalutaion of Intranasal Naltrexone

A single-center, open-label, randomized, four-sequence, four-treatment, four-period crossover pilot study was completed in healthy male and nonpregnant female subjects. Twenty (20) healthy subjects were enrolled. Twenty-one (20) subjects completed the study and have evaluable data for all study periods.

Endpoints. The primary study endpoint was to determine the pharmacokinetics of three different doses (1.2 mg, 1.6 mg, and 3 mg) of naltrexone hydrochloride nasal spray (Test Products 1, 2, and 3, respectively, also referred to herein as T1, T2, and T3) compared to a 50mg oral dose of naltrexone hydrochloride (Reference Product, also referred to herein as R) and to identify the intranasal dose of Test Product that could achieve naltrexone systemic exposure comparable to the 50 mg oral dose. The secondary study endpoint was to assess the safety and tolerability of the Test Product, especially nasal irritation (e.g., erythema, edema and erosion).

Inclusion and Exclusion Criteria. Inclusion criteria for the study were: written informed consent; male or female subject between 18 and 55 years, inclusive, at the time of consent; body weight ≥48 kg and body mass index (BMI) of 18.0 to 30.0 kg/m², inclusive; resting systolic blood pressure between 90 and 140 mmHg, inclusive, and diastolic blood pressure between 65 and 90, inclusive; resting heart rate between 40 and 100 bpm, inclusive; resting respiratory rate between 8 and 20, inclusive; adequate venous access; no clinically significant abnormalities on physical examination; no clinically significant abnormalities on 12-lead ECG, recorded after at least 3 minutes in supine position; no clinically significant abnormalities on hematology, biochemistry, coagulation and urinalysis parameters; negative test results for anti-HIV-1Ab and anti-HIV-2Ab antibodies, hepatitis B surface antigen (HBsAg) and anti-hepatitis C virus antibodies (anti-HCVAb); non-smoker or ex-smoker (i.e. someone who abstained from using tobacco-or nicotine-containing products within the previous 3 months; (occasional use of tobacco-or nicotine-containing products is acceptable, providing that there is no nicotine addiction and the subject agrees to abstain from smoking during the study periods); willingness to accept and comply with study restrictions (e.g. alcohol consumption, methylxanthines, diet, exercise, contraception and medications); and if female, infertile, postmenopausal, or if of childbearing potential, agrees to use an effective non-hormonal contraceptive or a hormonal contraceptive method from at least 4 weeks prior to admission to Period 1, and to continue on a stable continuous regimen until the end of the study to ensure stable plasma hormonal levels during the whole study duration.

Exclusion criteria at screening were: known hypersensitivity/allergy reaction to the study drug substance or any of the excipients; known severe hypersensitivity reaction to any other drug; any nasal conditions including abnormal nasal anatomy, nasal symptoms (i.e. blocked and/or runny nose, nasal polyps, etc.), rhinitis and other conditions that are known to impact nasal absorption or having a product sprayed in to the nasal cavity prior to drug administration; previous or current opioid, alcohol, or other drug dependence (excluding nicotine and caffeine), based on medical history; concurrent disease considered by the investigator to be clinically significant in the context of the study; need for concomitant treatment medication during the study; any medical condition (e.g. gastrointestinal, renal or hepatic, including peptic ulcer, inflammatory bowel disease or pancreatitis) or surgical condition (e.g. cholecystectomy, gastrectomy) that could affect drug pharmacokinetics (absorption, distribution, metabolism or excretion) or subject safety; current or recent (within 7 days prior to screening) upper respiratory tract infection; QT interval ≥450 msec for males and >470 msec for females; positive result in urine drugs-of-abuse test, or ethanol breath test; use of a depot injection or an implant of any drug (all but contraceptives) within the previous 3 months; average weekly alcohol consumption of >14 units (12 grams per unit) for males and >7 units for females within the previous 6 months; average daily consumption of methylxanthines-containing beverages or food (e.g. coffee, tea, cola, sodas, chocolate) equivalent to >500 mg methylxanthines (100 mg of methylxanthines is equivalent to approximately 150 mL of coffee, 300 mL of tea, 75 mL of hot chocolate, 800 mL of cola, 300mL of energy drinks, or 25 g chocolate bar); participation in any clinical trial within the previous 2 months, or in more than 2 clinical trials within the previous 12 months; blood donation or significant blood loss (≥450 mL) due to any reason or had plasmapheresis within the previous 2 months; difficulty in fasting or any dietary restriction such as lactose intolerance, vegan, low-fat, low sodium, etc., that may interfere with the diet served during the study; difficulty in donating blood on either arm; difficulty in swallowing capsules or tablets; if female, pregnant or breast-feeding; and any other condition that the Investigator considers rendering the subject unsuitable for the study.

Exclusion criteria at admission were: resting systolic blood pressure between 90 and 140 mmHg, inclusive, and diastolic blood pressure between 65 and 90, inclusive; resting heart rate between 40 and 100 bpm, inclusive; resting respiratory rate between 8 and 20, inclusive; QTcF interval >500 msec; significant arrhythmia defined as ≥6 beats of supraventricular tachycardia or ≥3 beats of ventricular tachycardia; any recent disease or condition or treatment that, according to the Investigator, would put the subject at undue risk due to study participation or occurred at a timeframe in which may interfere with the pharmacokinetics of study drug; use of prescription or nonprescription medicinal products, including vitamins, food supplements, herbal supplements (including St John's Wort), within the previous 14 days, unless in the Investigator's opinion the medication would not interfere with the pharmacokinetics of study drug or compromise subject safety (the use of topical products without systemic absorption, or recommended contraceptives was acceptable); consumption of any alcoholic product within the previous 72 hours; positive result in urine drugs-of-abuse test, or ethanol breath test; if female of childbearing potential, positive result in urine beta-hCG pregnancy test; and any other condition that the investigator considered rendering the subject unsuitable for the study period.

Clinical Procedure. Subjects were confined at the clinical research facilities for the duration of the study, 15 nights. Each subject underwent four treatments periods during the study. Each subject received one of the 4 treatments in each of the 4 treatment periods:

TABLE 8

| | |
|---|---|
| Reference (R) | One naltrexone hydrochloride (NTX) 50 mg film-coated tablets as a |
| | single dose. |
| Test Product 1 | Two administrations of 1.2 mg naltrexone hydrochloride (NTX) |
| (T1) | intranasal dose (two 0.1 mL spray of a 12 mg/mL solution), separated by |
| | two hours interval. T1 was prepared as set forth above in Formulation |
| | Example 49. |
| Test Product 2 | Two administrations of 1.6 mg naltrexone hydrochloride (NTX) |
| (T2) | intranasal dose (two 0.1 mL spray of a 16 mg/mL solution), separated by |
| | two hours interval. T2 was prepared as set forth above in Formulation |
| | Example 50. |
| Test Product 3 | Two administrations of 3 mg naltrexone hydrochloride (NTX) |
| (T3) | intranasal dose (two 0.1 mL spray of a 30 mg/mL solution), separated by |
| | two hours interval. T3 was prepared as set forth above in Formulation |
| | Example 52. |

According to the randomization schema, subjects were assigned to one of several treatment sequences in which investigational products T1, T2, T3, and R were administered in various orders. Investigational products were administered in the morning, after an overnight fasting of at least 10 hours. The Reference Product was administered as a single dose, orally, with 240 mL of water (swallowed). Each Test Product was administered twice: the first administration was performed in one nostril and the second was performed in the other nostril, 2 hours after, with the subject in an upright position. Subjects were instructed to hold their breath during administration of the nasal spray into the scheduled nostril. Test Product was primed before use. Following priming, the dosing device was weighed before and after each administration to determine the weight of the dose administered. Doses of each investigational product were separated by a washout interval of 3 days.

In each study period, subjects fasted overnight remained fasted until breakfast, which occurred approximately 3 hours after the Reference Product dosing and 1 hour after the second administration of Test Product dosing. No fluids were allowed from 1 hour before dose until 3 hours post-dose (timepoints defined in relation to first administration of Test product or ad ministration of Reference product). Water was provided ad libitum at all other times. Standardized meals and snacks identical in all periods were provided. Lunch was served approximately 3 hours after breakfast, and all other meals were scheduled at appropriate times by the clinical site.

Sample Collection and Analysis. In each study period, 24 venous blood samples (volume of 4 mL each) were collected for the determination of plasma concentrations of naltrexone and 6β-naltrexol at the following timepoints: pre-dose and at 0:02, 0:05, 0:10, 0:15, 0:20, 0:30, 0:45, 1:00, 2:00, 2:05, 2:10, 2:15, 2:20, 2:30, 2:45, 3:00, 4:00, 5:00, 6:00, 8:00, 12:00, 24:00 and 48:00 hours: minutes following Reference product dose and first dose of Test product.

Naltrexone and 6β-naltrexol plasma concentrations were measured using a previously validated liquid chromatography with tandem mass spectrometry (LC-MS/MS) analytical method.

Pharmacokinetic parameters of naltrexone and 6β-naltrexol were estimated with Phoenix® WinNonlin® version 8.1 (Certara USA Inc, Princeton, NJ) or higher, by using a non-compartmental approach with a ln-linear terminal phase assumption. Actual times of blood sampling was used to estimate pharmacokinetic parameters.

The following pharmacokinetic parameters were estimated: maximum observed plasma concentration (Cₘₐₓ); time of occurrence of Cₘₐₓ (tₘₐₓ); area under the plasma concentration versus time curve (AUC) from pre-dose (time zero) to the last sampling time with quantifiable concentrations (AUC₀₋ₜ); AUC from time zero to infinity (AUC_{0-∞}); apparent terminal elimination rate constant (λ_{z}); apparent terminal elimination half-life (t_{1/2}; though it should be noted that here, a single value is given for each two-dose administration) and apparent total clearance of the drug from plasma (CL/F). Cₘₐₓ, tₘₐₓ, AUC₀₋ₜ and AUC_{0-∞} of naltrexone and 6β-naltrexol were the primary endpoints.

Using a mixed effects model, an analysis of variance (ANOVA) was performed for comparisons of ln-transformed Cₘₐₓ, AUC₀₋ₜ and AUC_{0-∞}. Sequence, period and treatment were included in the ANOVA model as the independent factors. The Test-to-Reference geometric least-square means ratio (GMR) and the corresponding 90% confidence interval (CI) was calculated for Cₘₐₓ, AUC₀₋ₜ and AUC_{0-∞}. The relative extent (Frel) of intranasal absorption (Test versus Reference) was estimated from the dose-corrected AUC₀₋ₜ. Tₘₐₓ was compared using a non-parametric test. The secondary endpoints were λ_{z}, t_{1/2} and CL/F.

Safety. Safety was evaluated through the assessment of adverse events, 12-lead ECG, vital signs, nasal cavity examination, weight measurement, smell test and clinical laboratory tests (see flow-chart). Adverse events were monitored throughout the study.

Results. Results are given below and in the accompanying Figures.

Table 9 presents the average of naltrexone primary pharmacokinetic parameters and respective summary statistics for twenty (20) subjects, following administration of Test Product 1, Test Product 2, Test Product 3, and Reference Product.

TABLE 9

| | | Cₘₐₓ | tₘₐₓ | AUC₀₋ₜ | AUC_{0-∞} |
|---|---|---|---|---|---|
| | | (ng/mL) | (h) | (ng.h/mL) | (ng.h/mL) |
| Test Product 1 | n | 20 | 20 | 20 | 20 |
| (1.2 mg of NTX) | Gₘₑₐₙ | 8.10 | 0.58 | 12.42 | 12.60 |
| | Aₘₑₐₙ | 8.90 | 1.18 | 13.35 | 13.54 |
| | SD | 3.86 | 1.05 | 4.75 | 4.81 |
| | CV (%) | 43.3 | 88.9 | 35.6 | 35.5 |
| Test Product 2 | n | 20 | 20 | 20 | 20 |
| (1.6 mg of NTX) | Gₘₑₐₙ | 9.22 | 0.60 | 15.19 | 15.46 |
| | Aₘₑₐₙ | 9.68 | 1.18 | 16.07 | 16.35 |
| | SD | 3.03 | 1.03 | 5.03 | 5.09 |
| | CV (%) | 31.3 | 88.1 | 31.3 | 31.2 |
| Test Product 3 | n | 20 | 20 | 20 | 20 |
| (3.0 mg of NTX) | Gₘₑₐₙ | 19.88 | 0.80 | 31.33 | 31.94 |
| | Aₘₑₐₙ | 21.01 | 1.39 | 32.57 | 33.13 |
| | SD | 7.23 | 1.01 | 9.02 | 8.91 |
| | CV (%) | 34.4 | 72.5 | 27.7 | 26.9 |
| Reference | n | 20 | 20 | 20 | 19 |
| Product | Gₘₑₐₙ | 9.31 | 0.85 | 23.15 | 23.81 |
| | Aₘₑₐₙ | 11.14 | 0.98 | 26.14 | 27.09 |
| | SD | 6.92 | 0.65 | 13.65 | 14.53 |
| | CV (%) | 62.1 | 66.3 | 52.2 | 53.6 |

Table 10 presents the average of naltrexone secondary pharmacokinetic parameters and respective summary statistics for twenty (20) subjects, following administration of Test 1 Product, Test 2 Product, Test 3 Product, and Reference Product.

TABLE 10

| | | pAUC_{0-0.033} | pAUC_{0-0.083} | pAUC_{0-0.167} | pAUC_{0-0.250} | pAUC_{0-0.333} | pAUC_{0-0.500} | λ_{z} | | CL/F |
|---|---|---|---|---|---|---|---|---|---|---|
| | | (ng · h/mL) | (ng · h/mL) | (ng · h/mL) | (ng · h/mL) | (ng · h/mL) | (ng · h/mL) | (1/h) | t_{1/2} (h) | (L/h) |
| Test 1 | n | 20 | 20 | 20 | 20 | 20 | 20 | 20 | 20 | 20 |
| Product | Gₘₑₐₙ | | 0.11 | 0.56 | 1.08 | 1.50 | 2.09 | 0.295 | 2.35 | 190.45 |
| (1.2 mg of | Aₘₑₐₙ | 0.02 | 0.16 | 0.66 | 1.22 | 1.67 | 2.28 | 0.324 | 2.64 | 207.51 |
| NTX) | SD | 0.04 | 0.10 | 0.34 | 0.56 | 0.70 | 0.88 | 0.129 | 1.43 | 95.66 |
| | CV (%) | 169.5 | 66.0 | 50.9 | 45.7 | 42.0 | 38.6 | 39.9 | 54.2 | 46.1 |
| Test 2 | n | 19 | 20 | 20 | 20 | 20 | 20 | 20 | 20 | 20 |
| Product | Gₘₑₐₙ | 0.08 | 0.50 | 1.03 | 1.47 | 2.11 | 0.219 | 3.16 | 206.97 | 0.08 |
| (1.6 mg of | Aₘₑₐₙ | 0.01 | 0.10 | 0.58 | 1.18 | 1.66 | 2.36 | 0.267 | 3.86 | 222.73 |
| NTX) | SD | 0.01 | 0.06 | 0.29 | 0.56 | 0.78 | 1.04 | 0.158 | 2.31 | 105.01 |
| | CV (%) | 105.1 | 60.1 | 49.2 | 48.0 | 46.8 | 44.1 | 59.3 | 59.8 | 47.1 |
| Test 3 | n | 20 | 20 | 20 | 20 | 20 | 20 | 20 | 20 | 20 |
| Product | Gₘₑₐₙ | 0.01 | 0.21 | 1.09 | 2.10 | 2.93 | 4.14 | 0.130 | 5.34 | 187.87 |
| (3.0 mg of | Aₘₑₐₙ | 0.02 | 0.27 | 1.26 | 2.41 | 3.35 | 4.69 | 0.137 | 6.06 | 195.47 |
| NTX) | SD | 0.02 | 0.17 | 0.67 | 1.21 | 1.61 | 2.12 | 0.036 | 4.93 | 58.78 |
| | CV (%) | 83.8 | 63.2 | 52.9 | 50.3 | 48.0 | 45.2 | 25.9 | 81.4 | 30.1 |
| Reference | n | 19 | 20 | 20 | 20 | 20 | 20 | 19 | 19 | 19 |
| Product | Gₘₑₐₙ | | | | | | | 0.127 | 5.45 | 2099.84 |
| | Aₘₑₐₙ | 0.00 | 0.00 | 0.00 | 0.04 | 0.16 | 0.79 | 0.130 | 5.57 | 2364.41 |
| | SD | 0.00 | 0.00 | 0.01 | 0.08 | 0.27 | 0.95 | 0.029 | 1.21 | 1121.61 |
| | CV (%) | | | 235.8 | 208.1 | 165.6 | 120.2 | 22.5 | 21.8 | 47.4 |

Table 11 presents the average naltrexone pharmacokinetic parameters and respective summary statistics for twenty (20) subjects, following first and second administrations of Test 1 Product, Test 2 Product, Test 3 Product, and Reference Product

TABLE 11

| | | 1^{st} Administration | | | 2^{nd} Administration | | |
|---|---|---|---|---|---|---|---|
| | Cₘₐₓ | | pAUC₀₋₂ | Cₘₐₓ | | pAUC₂₋ₜ | Cₘₐₓ |
| | (ng/mL) | tₘₐₓ (h) | (ng · h/mL) | (ng/mL) | tₘₐₓ (h) | (ng · h/mL) | (ng/mL) |
| Test 1 | n | 20 | 20 | 20 | 20 | 20 | 20 |
| Product | Gₘₑₐₙ | 6.98 | 0.17 | 4.34 | 5.64 | 2.19 | 7.69 |
| (1.2 mg | Aₘₑₐₙ | 7.87 | 0.18 | 4.64 | 6.87 | 2.19 | 8.67 |
| of NTX) | SD | 3.61 | 0.06 | 1.57 | 3.97 | 0.06 | 3.73 |
| | CV (%) | 45.9 | 34.9 | 33.9 | 57.8 | 2.7 | 43.1 |
| Test 2 | n | 20 | 20 | 20 | 20 | 20 | 20 |
| Product | Gₘₑₐₙ | 7.25 | 0.18 | 4.69 | 7.72 | 2.19 | 10.11 |
| (1.6 mg | Aₘₑₐₙ | 8.12 | 0.18 | 5.11 | 8.47 | 2.19 | 10.92 |
| of NTX) | SD | 3.74 | 0.03 | 1.95 | 2.87 | 0.05 | 3.66 |
| | CV (%) | 46.1 | 18.6 | 38.3 | 33.9 | 2.2 | 33.5 |
| Test 3 | n | 20 | 20 | 20 | 20 | 20 | 20 |
| Product | Gₘₑₐₙ | 13.98 | 0.16 | 8.89 | 15.15 | 2.21 | 21.36 |
| (3.0 mg | Aₘₑₐₙ | 16.07 | 0.18 | 9.99 | 17.13 | 2.21 | 22.50 |
| of NTX) | SD | 8.15 | 0.08 | 4.14 | 8.21 | 0.06 | 7.08 |
| | CV(%) | 50.7 | 43.4 | 41.4 | 47.9 | 2.6 | 31.4 |
| Reference | n | 19 | 20 | 20 | 20 | 20 | 20 |
| Product | Gₘₑₐₙ | | | | 0.127 | 5.45 | 2099.84 |
| | Aₘₑₐₙ | 0.00 | 0.00 | 0.00 | 0.04 | 0.16 | 0.79 |
| | SD | 0.00 | 0.00 | 0.01 | 0.08 | 0.27 | 0.95 |
| | CV (%) | | 235.8 | 208.1 | 165.6 | 120.2 | 22.5 |

Table 12 presents the average of 6β-naltrexol primary pharmacokinetic parameters and respective summary statistics for twenty (20) subjects, following administration of Test 1 Product, Test 2 Product, Test 3 Product, and Reference Product.

TABLE 12

| | | Cₘₐₓ | tₘₐₓ | AUC₀₋ₜ | AUC_{0-∞} |
|---|---|---|---|---|---|
| | | (ng/mL) | (h) | (ng.h/mL) | (ng.h/mL) |
| Test 1 | n | 20 | 20 | 20 | 20 |
| Product | Gₘₑₐₙ | 2977.69 | 2.43 | 39661.64 | 44752.81 |
| (1.2 mg | Aₘₑₐₙ | 4726.08 | 3.00 | 43010.42 | 49049.12 |
| of NTX) | SD | 8815.95 | 1.65 | 18372.27 | 22391.88 |
| | CV (%) | 186.5 | 55.0 | 42.7 | 45.7 |
| Test 2 | n | 20 | 20 | 20 | 20 |
| Product | Gₘₑₐₙ | 3460.35 | 2.93 | 45308.65 | 51158.30 |
| (1.6 mg | Aₘₑₐₙ | 5772.44 | 3.10 | 49901.89 | 57840.89 |
| of NTX) | SD | 11486.17 | 0.88 | 24055.17 | 34255.04 |
| | CV (%) | 199.0 | 28.4 | 48.2 | 59.2 |
| Test 2 | n | 20 | 20 | 20 | 20 |
| Product | Gₘₑₐₙ | 5423.19 | 3.03 | 77062.89 | 86359.93 |
| (3.0 mg | Aₘₑₐₙ | 5742.45 | 3.09 | 80141.96 | 89628.51 |
| of NTX) | SD | 1885.91 | 0.64 | 22003.15 | 24149.84 |
| | CV (%) | 32.8 | 20.8 | 27.5 | 26.9 |
| Reference | n | 20 | 20 | 20 | 20 |
| Product | Gₘₑₐₙ | 111377.95 | 0.97 | 727323.16 | 795385.17 |
| | Aₘₑₐₙ | 117199.63 | 1.13 | 739175.55 | 809654.98 |
| | SD | 37154.79 | 0.71 | 129584.07 | 150316.07 |
| | CV (%) | 31.7 | 62.7 | 17.5 | 18.6 |

Table 13 presents the average of 6β-naltrexol secondary pharmacokinetic parameters and respective summary statistics for twenty (20) subjects, following administration of Test 1 Product, Test 2 Product, Test 3 Product, and Reference Product.

TABLE 13

| | | pAUC_{0-0.033} | pAUC_{0-0.083} | pAUC_{0-0.167} | pAUC_{0-0.250} | pAUC_{0-0.333} | PAUC_{0-0.500} | λ_{z} | | CL/F |
|---|---|---|---|---|---|---|---|---|---|---|
| | | (ng · h/mL) | (ng · h/mL) | (ng · h/mL) | (ng · h/mL) | (ng · h/mL) | (ng · h/mL) | (1/h) | t_{1/2} (h) | (L/h) |
| Test | n | 20 | 20 | 20 | 20 | 20 | 20 | 20 | 20 | 20 |
| Product 1 | Gₘₑₐₙ | | | 52.32 | 121.46 | 218.83 | 450.19 | 0.045 | 15.43 | 53.63 |
| (1.2 mg of | Aₘₑₐₙ | 17.83 | 39.49 | 94.17 | 174.39 | 282.58 | 531.05 | 0.046 | 15.75 | 58.42 |
| NTX) | SD | 26.38 | 55.90 | 111.62 | 170.14 | 233.62 | 352.46 | 0.009 | 3.41 | 24.11 |
| | CV (%) | 147.9 | 141.5 | 118.5 | 97.6 | 82.7 | 66.4 | 19.4 | 21.6 | 41.3 |
| Test | n | 19 | 20 | 20 | 20 | 20 | 20 | 20 | 20 | 20 |
| Product 2 | Gₘₑₐₙ | | | 49.63 | 116.10 | 208.74 | 434.59 | 0.047 | 14.85 | 62.55 |
| (1.6 mg of | Aₘₑₐₙ | 14.45 | 35.67 | 87.27 | 166.76 | 273.69 | 530.56 | 0.048 | 15.28 | 70.05 |
| NTX) | SD | 20.65 | 50.66 | 102.20 | 159.50 | 225.54 | 368.09 | 0.010 | 4.17 | 36.83 |
| | CV (%) | 143.0 | 142.0 | 117.1 | 95.6 | 82.4 | 69.4 | 20.9 | 27.3 | 52.6 |
| Test | n | 20 | 20 | 20 | 20 | 20 | 20 | 20 | 20 | 20 |
| Product 3 | Gₘₑₐₙ | | | 58.40 | 153.54 | 294.84 | 647.77 | 0.047 | 14.59 | 69.48 |
| (3.0 mg of | Aₘₑₐₙ | 9.93 | 27.32 | 82.31 | 191.08 | 353.70 | 750.71 | 0.048 | 14.93 | 72.36 |
| NTX) | SD | 13.68 | 33.68 | 69.66 | 127.72 | 212.93 | 392.87 | 0.009 | 3.57 | 22.31 |
| | CV (%) | 137.8 | 123.3 | 84.6 | 66.8 | 60.2 | 52.3 | 19.0 | 23.9 | 30.8 |
| Reference | n | 19 | 20 | 20 | 20 | 20 | 20 | 20 | 20 | 20 |
| Product | Gₘₑₐₙ | | | | | 273.59 | 2489.10 | 0.048 | 14.39 | 62.86 |
| | Aₘₑₐₙ | 2.11 | 5.70 | 49.31 | 427.56 | 1769.35 | 8799.34 | 0.049 | 14.67 | 64.10 |
| | SD | 2.90 | 7.39 | 75.91 | 713.22 | 2729.39 | 9755.51 | 0.010 | 2.97 | 13.57 |
| | CV (%) | 137.7 | 129.6 | 153.9 | 166.8 | 154.3 | 110.9 | 19.5 | 20.2 | 21.2 |

In accordance to the protocol, using a mixed effects model, an analysis of variance (ANOVA) was performed for comparisons of naltrexone ln-transformed Cₘₐₓ, AUC₀₋ₜ and AUC_{0-∞}. The Test-to-Reference geometric least-square means ratio (GMR) and the corresponding 90% confidence interval (CI) was calculated. Results comparing the three different Test Products (Test Product 1, Test Product 2, and Test Product 3) with Reference are summarized in Table 14.

TABLE 14
Mixed Effects ANOVA results for
Naltrexone Pharmacokinetic Parameters.

| | | | Test 1-to- | |
|---|---|---|---|---|
| | Geometric LSmeans¹ | | Reference | |
| PK Parameter | Test 1 | Reference | GMR (%) | 90% CI |
| Cₘₐₓ | 8.10 | 9.31 | 86.93 | 65.69-115.04 |
| AUC₀₋ₜ | 12.42 | 23.15 | 53.63 | 41.69-69.00 |
| AUC_{0-∞} | 12.59 | 23.80 | 52.92 | 41.15-68.05 |
| | | | Test 2-to- | |
| | | | Reference | |
| PK Parameter | Test 2 | Reference | GMR (%) | 90% CI |
| Cₘₐₓ | 9.22 | 9.31 | 99.00 | 74.54-131.49 |
| AUC₀₋ₜ | 15.19 | 23.15 | 65.63 | 50.98-84.49 |
| AUC_{0-∞} | 15.42 | 23.80 | 64.78 | 50.34-83.37 |
| | | | Test 3-to- | |
| | | | Reference | |
| PK Parameter | Test 3 | Reference | GMR (%) | 90% CI |
| Cₘₐₓ | 19.88 | 9.31 | 213.41 | 162.41-280.42 |
| AUC₀₋ₜ | 31.33 | 23.15 | 135.34 | 107.88-169.80 |
| AUC_{0-∞} | 31.85 | 23.80 | 133.83 | 106.74-167.80 |

¹LSmeans values are given in ng/mL for Cₘₐₓ and ng.h/mL for AUC.

In accordance to the protocol, using a mixed effects model, an analysis of variance (ANOVA) was performed for comparisons of 6β-naltrexol ln-transformed Cₘₐₓ, AUC₀₋ₜ and AUC_{0-∞}. The Test-to-Reference geometric least-square means ratio (GMR) and the corresponding 90% confidence interval (CI) was calculated. Results comparing the three different Test Products (Test 1, Test 2, and Test 3) with Reference are summarized in Table 15.

TABLE 15
Mixed Effects ANOVA results for
Naltrexone Pharmacokinetic Parameters.

| | | | Test 1-to- | |
|---|---|---|---|---|
| | Geometric LSmeans | | Reference | |
| PK Parameter | Test 1 | Reference | GMR (%) | 90% CI |
| Cₘₐₓ | 2977.69 | 111377.95 | 2.67 | 2.01-3.56 |
| AUC₀₋ₜ | 39661.64 | 727323.16 | 5.45 | 4.75-6.26 |
| AUC_{0-∞} | 44752.81 | 795385.17 | 5.63 | 4.91-6.44 |
| | | | Test 2-to- | |
| | | | Reference | |
| PK Parameter | Test 2 | Reference | GMR (%) | 90% CI |
| Cₘₐₓ | 3460.35 | 111377.95 | 3.11 | 2.34-4.12 |
| AUC₀₋ₜ | 45308.65 | 727323.16 | 6.23 | 5.38-7.21 |
| AUC_{0-∞} | 51158.30 | 795385.17 | 6.43 | 5.51-7.51 |
| | | | Test 3-to- | |
| | | | Reference | |
| PK Parameter | Test 3 | Reference | GMR (%) | 90% CI |
| Cₘₐₓ | 5423.19 | 111377.95 | 4.87 | 4.06-5.83 |
| AUC₀₋ₜ | 77062.89 | 727323.16 | 10.60 | 9.39-11.95 |
| AUC_{0-∞} | 86359.93 | 795385.17 | 10.86 | 9.67-12.20 |

¹LSmeans values are given in ng/mL for Cmax and ng.h/mL for AUC

In accordance to the protocol, the relative extent (Fᵣₑₗ) of intranasal absorption (Test versus Reference) of naltrexone was estimated from the dose-corrected AUC₀₋ₜ (AUC_{0-t/D}) of the drug. Table 16 presents the arithmetic means and coefficient of variation (CV %) for the estimated parameters.

TABLE 16
Naltrexone-Relative Extent of Intranasal Absorption

| PK Parameter | Test 1 | Test 2 | Test 3 | Reference |
|---|---|---|---|---|
| (unit) | (n =20) | (n =20) | (n = 20) | (n = 20) |
| AUC₀₋ₜ | 13.35 (35.6%) | 16.07 (31.3%) | 32.57 (27.7%) | 26.14 (52.2%) |
| (ng.h/mL) | | | | |
| AUC₀₋ₜ/D | 5.56 (35.6%) | 5.02 (31.3%) | 5.43 (27.7%) | 0.52 (52.2%) |
| (ng.h/mL/mg) | | | | |
| Fᵣₑₗ (%) | 1328.78 (61.7%) | 1238.87 (65.8%) | 1306.38 (61.9%) | |

n-Number of Subjects
AUC₀₋ₜ/D-Dose normalized AUC0-t. For each Test product, D corresponds to the total administered dose of the product
Fᵣₑₗ-relative extent of intranasal absorption (Test/Reference)

In accordance to the protocol, tmax obtained for each Formulation was compared using a non-parametric test as shown in Table 17.

TABLE 17
Naltrexone: tₘₐₓ Non-parametric Analysis.

| Treatment Difference | Median | 90% CI |
|---|---|---|
| Test 1-Reference | 0.29 | −0.54-0.54 |
| Test 2-Reference | 0.29 | −0.46-0.64 |
| Test 3-Reference | 0.46 | 0.17-1.17 |

### Other Embodiments

The detailed description set forth herein is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references, patents or applications, U.S. or foreign, cited in the application are hereby incorporated by reference as if written herein in their entireties. Where any inconsistencies arise, material literally disclosed herein controls.

Although the invention has been described with reference to the above examples, it will be understood that modifications and variations are encompassed within the spirit and scope of the invention. Accordingly, the invention is limited only by the following claims.

1. An intranasal formulation comprising an aqueous solution comprising between about 1 mg and about 4 mg naltrexone, or a pharmaceutically acceptable salt thereof.

2. An intranasal formulation comprising, in a volume of about 50 to about 150 μL, an aqueous solution comprising between about 10 mg/mL and about 40 mg/mL naltrexone, or a pharmaceutically acceptable salt thereof.

3. The formulation as recited in claim 1, additionally comprising:
an isotonicity agent;
a preservative;
a stabilizing agent;
an absorption enhancer; and
an amount of water sufficient to achieve a final volume of about 50 to about 150 μL.

4. The formulation as recited in claim 3, comprising:
between about 1 mg and about 3 mg naltrexone or a pharmaceutically acceptable salt thereof;
between about 0.1 mg and about 1.2 mg of the isotonicity agent;
between about 0.001 mg and about 0.1 mg of the preservative;
between about 0.1 mg and about 0.5 mg of the stabilizing agent;
between about 0.05 mg and about 2.5 mg of the absorption enhancer; and
an amount of water sufficient to achieve a final volume of about 50 to about 150 μL.

5. The formulation as recited in claim 3, comprising:
between about 1% and about 3% naltrexone or a pharmaceutically acceptable salt thereof;
between about 0.1% and about 1.2% of the isotonicity agent;
between about 0.001% and about 0.1% of the preservative;
between about 0.1% and about 0.5% of the stabilizing agent;
between about 0.05% mg and about 2.5% of the absorption enhancer.

6. The formulation as recited in claim 4, wherein:
the isotonicity agent is NaCl;
the preservative is benzalkonium chloride;
the stabilizing agent is disodium edetate; and
the absorption enhancer is an alkylsaccharide.

7. The formulation as recited in claim 6, wherein the alkylsaccharide is dodecyl maltoside.

8. The formulation as recited in claim 7, comprising:
between about 1 mg and about 3 mg naltrexone or a pharmaceutically acceptable salt thereof;
between about 0.1 mg and about 1.2 mg of NaCl;
between about 0.001 mg and about 0.1 mg of benzalkonium chloride;
between about 0.15 mg and about 0.5 mg of disodium edetate;
between about 0.05 mg and about 2.5 mg of dodecyl maltoside; and
an amount of water sufficient to achieve a final volume of about 50 to about 150 μL.

9. The formulation as recited in claim 8, comprising between about 0.1 mg to about 0.5 mg of dodecyl maltoside.

10. The formulation as recited in claim 9, comprising about 0.25 mg of dodecyl maltoside.

11. The formulation as recited in claim 8, comprising about 0.2 mg and about 0.3 mg of disodium edetate.

12. The formulation as recited in claim 9, comprising:
between about 1 mg and about 3 mg naltrexone or a pharmaceutically acceptable salt thereof;
between about 0.3 mg and about 0.7 mg of NaCl;
about 0.02 mg of benzalkonium chloride;
about 0.3 mg of disodium edetate;
about 0.25 mg of dodecyl maltoside; and
an amount of water sufficient to achieve a final volume of about 50 to about 150 μL.

13. The formulation as recited in claim 10, wherein the amount of water is sufficient to achieve a final volume of about 80 to about 120 μL.

14. The formulation as recited in claim 11, wherein the amount of water is sufficient to achieve a final volume of about 100 μL.

15. The formulation as recited in claim 7, comprising:
between about 1% and about 3% naltrexone or a pharmaceutically acceptable salt thereof;
between about 0.1% and about 1.2% of NaCl;
between about 0.001% and about 0.1% of benzalkonium chloride;
between about 0.15% and about 0.5% of disodium edetate;
between about 0.05% and about 2.5% of dodecyl maltoside; and
water.

16. The formulation as recited in claim 15, comprising between about 0.1% to about 0.5% of dodecyl maltoside.

17. The formulation as recited in claim 16, comprising about 0.25% of dodecyl maltoside.

18. The formulation as recited in claim 15, comprising about 0.2% and about 0.3% of disodium edetate.

19. The formulation as recited in claim 17, comprising:
between about 1% and about 3% naltrexone or a pharmaceutically acceptable salt thereof;
between about 0.3% and about 0.7% of NaCl;
about 0.02% of benzalkonium chloride;
about 0.3% of disodium edetate;
about 0.25% of dodecyl maltoside; and
water.

20. The formulation as recited in claim 19, wherein the amount of water is sufficient to achieve a final volume of about 50 to about 150 μL.

21.-66. (canceled)
